(19) United States
(12) Patent Application Publication
(10) Pub. No.: US 2025/0262190 A1
(43) Pub. Date: Aug. 21, 2025

(54) USE OF LAT1 INHIBITORS TO TREAT OBESITY

(71) Applicant: Georgetown University, Washington, DC (US)

(72) Inventors: Jeffrey Huang, Bethesda, MD (US); Mi-Hye Lee, Rockville, MD (US); Maryna Baydyuk, Springfield, VA (US)

(73) Assignee: Georgetown University, Washington, DC (US)

(21) Appl. No.: 18/859,396

(22) PCT Filed: Apr. 24, 2023
(86) PCT No.: PCT/US2023/019686

§ 371 Date: Oct. 23, 2024

Related U.S. Application Data
(60) Provisional application No. 63/334,624, filed on Apr. 25, 2022.

(51) Int. Cl. A61K 31/423; A61P 3/04

(52) U.S. Cl. CPC A61K 31/423; A61P 3/04

(57) ABSTRACT

Methods of treating obesity, reducing body weight, or inhibiting weight gain in a subject in need thereof, the methods comprising administering to the subject a pharmaceutical composition comprising an effective amount of a large amino acid transporter small subunit1 (LAT1) inhibitor. The LAT1 inhibitor may be a small molecule, such as JPH203 or OKY-034.

### CROSS-REFERENCE TO RELATED APPLICATIONS

This application claims the benefit of U.S. Provisional Application No. 63/334,624, filed on Apr. 25, 2022, which is incorporated herein by reference in its entirety.

### BACKGROUND OF THE INVENTION

Adipocytes, or fat cells, serve as a sink for lipid storage in our bodies (Church et al., 2012). However, when lipid accumulation far exceeds their breakdown, adipocytes, in response will enlarge, expand, and ultimately recruit immune cells to adipose tissues and promote inflammation (Church et al., 2012). This pathological metabolic response can lead to obesity and chronic health problems if not regulated.

Obesity is a major risk factor of type 2 diabetes, stroke, heart disease, certain cancers, and age-related diseases, and its prevalence is growing across the world every year (Ades & Savage, 2017; Grundy et al., 2005; Roberts et al., 2010). Other than modifying lifestyle and diet, which can be difficult to maintain for many adults, there are no effective drugs to stop the metabolic response to excess lipid intake in adipocytes.

Therefore, strategies to temper the metabolic response could effectively restore adipocyte homeostasis, and prevent the development of obesity and associated diseases.

### SUMMARY OF THE INVENTION

The present application is directed to methods and compositions for treating obesity or reducing weight or fat mass in a subject.

In one aspect, the present invention relates to a method of treating obesity in a subject in need thereof. In another aspect, the present invention relates to a method of reducing body weight in a subject in need thereof. In yet another aspect, the present invention relates to a method of inhibiting weight gain in a subject in need thereof. In a further aspect, the present invention relates to a method of reducing white adipose tissue in a subject in need thereof. In an additional aspect, the present invention relates to a method of preventing an increase in white adipose tissue in a subject in need thereof. In yet another aspect, the present invention relates to a method of slowing or preventing the development of diet-associated metabolic syndrome in a subject in need thereof.

The methods of the present invention may comprise administering to the subject a pharmaceutical composition comprising an effective amount of a large amino acid transporter small subunit1 (LAT1) inhibitor.

In some embodiments, the obesity, weight gain, or increase in white adipose tissue is diet-associated. In certain embodiments, the obesity, weight gain, or increase in white adipose tissue is associated with a high-fat diet.

In some embodiments, the reduction in white adipose tissue or the prevention in the increase in white adipose tissue is in epididymal white adipose tissue, inguinal white adipose tissue, or a combination thereof.

In some embodiments, the slowing or prevention in the development of metabolic syndrome comprises a slowing or prevention in the development of metabolic disorders associated with metabolic syndrome. In certain embodiments, the metabolic disorder is dyslipidemia, hypertension, impaired glucose tolerance, compensatory hyperinsulinemia, the tendency to develop fat around the abdomen, or a combination thereof.

In embodiments of the invention, the pharmaceutical composition is administered systemically. In certain embodiments, the route of systemic administration is selected from intravenously, orally, intraperitoneally, intramuscularly, intradermally, intrathecally, subcutaneously and nasally.

In some embodiments, the pharmaceutical composition is administered locally to adipose tissue.

In some embodiments, the subject is a mammal. In certain embodiments, the mammal is human.

In another aspect, the present invention relates to a method of reducing expression of leptin in an adipocyte. The method comprises contacting the adipocyte with a LAT1 inhibitor.

In yet another aspect, the present invention relates to a method of reducing inflammation in adipose tissue. The method comprises contacting the adipose tissue with a LAT1 inhibitor.

In a further aspect, the present invention relates to a method of inducing or increasing expression of one or more brown adipose tissue markers in white adipose tissue. The method comprises contacting the white adipose tissue with a LAT1 inhibitor. In some embodiments, the one or more brown adipose tissue markers is selected from the group consisting of uncoupling protein 1, transmembrane protein 26, fibroblast growth factor 21, peroxisome proliferator-activated receptor gamma co-activator-1 alpha, and any combination thereof.

In another aspect, the present invention relates to a method of inducing conversion of white adipose tissue to brown adipose tissue. The method comprises contacting the white adipose tissue with a LAT1 inhibitor.

In some embodiments of the invention, the LAT1 inhibitor is JPH203 or OKY-034. In some embodiments, the LAT1 inhibitor is modified to promote the LAT1 inhibitor to target or enter adipose tissue, white adipose tissue, or an adipocyte.

In another aspect, the present invention relates to a LAT1 inhibitor or pharmaceutical composition thereof, for use in any of the methods of the invention.

These and other aspects of the present invention are described further in the below Detailed Description, Drawings, Examples and Claims sections of this patent disclosure. Furthermore, one of skill in the art will recognize that the various embodiments of the present invention described throughout this patent disclosure can be combined in various different ways, and that such combinations are within the scope of the present invention.

### BRIEF DESCRIPTION OF THE DRAWINGS

FIG. 1 shows upregulation of Slc7a5 in fully differentiated adipocyte and adipose fat tissues in high fat diet (HFD)-fed mice as compared to normal diet-fed mice, as described in Example 1. Panel A shows Slc7a5 mRNA expression by quantitative reverse transcription polymerase chain reaction (RT-qPCR) in cell culture system. Panel B shows Slc7a5 mRNA expression by RT-qPCR in epididymal white adipose tissues of mice under normal- vs HFD-fed conditions. Panels C and D show quantification of Slc7a5 mRNA levels by RNAscope in epididymal adipose tissue and inguinal adipose tissue, respectively, of normal- vs HFD-fed mice. Panel E shows representative images of coimmunostaining analysis, which exhibits Slc7a5 mRNA puncta (red) in leptin+adipocyte (green) and/or Iba1+microglia/macrophages (pink) (Zeiss confocal LSM800 63× oil lens). [“Pre L1”=Preadipocyte 3T3-L1; “Dif L1”=Differentiated 3T3-L1; “Epi fat”=epididymal white adipose tissue; “SubQ”=inguinal white adipose tissue; data shown as mean±standard error of the mean (SEM) and significance was determined by unpaired two-tailed t-test; *P<0.05; **P<0.01; ***P<0.001.]

FIG. 2 shows the effect of JPH203 treatment on body composition and food intake in HFD-fed mice, as described in Example 2. Panels A and B show body weight and % body weight over 15 days, respectively, of normal diet-fed mice, HFD-fed mice injected with JPH203, and control HFD-fed mice injected with phosphate buffer solution (PBS). Panel C shows the mass at day 15 of interscapular brown adipose tissue, epididymal white adipose tissue, and inguinal white adipose tissue of normal diet-fed mice, HFD-fed mice injected with JPH203, and control HFD-fed mice. Panels D and E show consumption of food (Panel D) and water (Panel E) over 15 days in HFD-fed mice injected with JPH203 and control HFD-fed mice. [“iBAT”=interscapular brown adipose tissue; “Epi fat”=epididymal white adipose tissue; “SubQ”=inguinal white adipose tissue; data shown as mean±SEM and significance was determined by unpaired two-tailed t-test (for analysis shown in Panels A and C) or multiple unpaired t-test (for analysis shown in Panels B, D, and E). *P<0.05, **P<0.01, ***P<0.001.

FIG. 3 shows the effect of JPH203 treatment on liver and kidney function, activity, and organs mass in HFD-fed mice, as described in Example 2. Panels A and B shows serum levels of aspartate aminotransferase (AST) (Panel A) and alanine aminotransferase (ALT) (Panel B) as indicators of liver function, and Panel C shows serum level of creatinine as an indicator of kidney function, in normal diet-fed mice, HFD-fed mice injected with JPH203, and control HFD-fed mice. Panel D shows average distance from a running wheel experiment as an indicator of the mice activity in normal diet-fed mice, HFD-fed mice injected with JPH203, and control HFD-fed mice. Panel E shows the mass at day 15 of the liver, heart, and kidney of mice fed a normal diet, HFD-fed mice injected with JPH203, and control HFD-fed mice.

FIG. 4 shows the effect of JPH203 treatment on bone marrow and immune response (whole blood counts) in HFD-fed mice, as described in Example 2. Panels A-S show measurements of white blood cells (WBC), red blood cells (RBC), hemoglobin (HGB), hematocrit (HCT), mean corpuscular volume (MCV), major histocompatibility complex (MCH), mean corpuscular hemoglobin concentration (MCHC), cell haemoglobin concentration mean (CHCM), mean cellular hemoglobin content (CH), red cell distribution width (RDW), hemoglobin distribution width (HDW), platelet (PLT), mean platelet volume (MPV), neutrophils, lymphocytes, monocytes, eosinophils, basophils, and leukocytes (LUC), respectively, in normal diet-fed mice, HFD-fed mice injected with JPH203, and control HFD-fed mice.

FIG. 5 shows the effect of JPH203 treatment on serum levels of adipokine and cytokines measured by enzyme-linked immunosorbent assay (ELISA), as described in Example 2. Panels A-G show serum levels of leptin, insulin like growth factor 1 (IGF1), monocyte chemoattractant protein-1 (MCP1), vascular endothelial growth factor (VEGF), interleukin 1 alpha (ILla), interleukin 1 beta (IL1β), interleukin 6, and tumor necrosis factor alpha (TNFa), respectively, in normal diet-fed mice, HFD-fed mice injected with JPH203, and control HFD-fed mice. [“AU”=arbitrary unit; data shown as mean±SEM and significance was determined by one-way analysis of variance (ANOVA) or unpaired two-tailed t-test; *P<0.05, **P<0.01; ***P<0.001, ****P<0.0001.]

FIG. 6 shows the effect of JPH203 treatment on adipocyte gene expression profile in epididymal white adipose tissue, as described in Example 2. Panels A-F show mRNA levels of general adipocyte markers leptin, homeobox C9 (hoxc9), peroxisome proliferator-activated receptors gamma (PPARγ), CCAAT enhancer binding protein alpha (CEBPα), sterol regulatory element-binding transcription factor 1c (SREBF1c), and adiponectin, respectively, of normal diet-fed mice, HFD-fed mice injected with JPH203, and control HFD-fed mice. Panel G shows mRNA level of white adipocyte marker Asc1 of normal diet-fed mice, HFD-fed mice injected with JPH203, and control HFD-fed mice. Panels H-K show mRNA levels of beiging or browning markers uncoupling protein 1 (UCP1), transmembrane protein 26 (Tmem26), fibroblast growth factor 21 (FGF21), and peroxisome proliferator-activated receptor gamma co-activator-1 alpha (PGC1α). Panels L-O show mRNA levels of proinflammatory inducible nitric oxide synthase (inos), anti-inflammatory arginase 1 (Arg1), Slc7a5, and hypoxia-inducible factor-2 alpha (Hif2α), respectively, of normal diet-fed mice, HFD-fed mice injected with JPH203, and control HFD-fed mice. [data shown as mean±SEM and significance was determined by one-way ANOVA; *P<0.05, ***P<0.001, ****P<0.0001.]

FIG. 7 shows the effect of JPH203 treatment on lipid homeostasis in epididymal white adipose tissue, as described in Example 2. Panels A and B show mRNA levels of genes related to glyceroneogenesis/fatty acid synthesis, acetyl-coA synthase (ACS) (Panel A) in and fatty acid synthase (FAS) (Panel B), of normal diet-fed mice, HFD-fed mice injected with JPH203, and control HFD-fed mice. Panel C shows mRNA level of lipogenesis-related gene diacylglycerol O-acyltransferase 1 (DGAT1), and Panel D shows mRNA level of pentose phosphate pathway-related gene glucose dehydrogenase (GDH), in normal diet, HFD-fed mice injected with JPH203, and control HFD-fed mice. Panel E shows mRNA level of lipolysis-related gene adipose triglyceride lipase (ATGL), and Panel F shows mRNA level of glucose uptake/glycolysis-related gene glucose transporter 4 (GLUT4), in normal diet, HFD-fed mice injected with JPH203, and control HFD-fed mice. Panel G shows mRNA level of fatty acid uptake-related gene fatty acid transport protein (FATP), and Panels H-L show mRNA levels of energy dissipation/fatty acid β oxidation-related genes acyl-coA dehydrogenase very long chain (ACADVL), acyl-coenzyme A oxidase (ACOX1), long chain acyl-coA dehydrogenase (LCAD), mitochondrial uncoupling protein 2 (UCP2), and peroxisome proliferator-activated receptor beta or delta (PPARB8), respectively, in normal diet, HFD-fed mice injected with JPH203, and control HFD-fed mice. Panel M shows mRNA level of anti-adipogenic factor-related gene C/EBP homologous protein 10 (CHOP10), and Panel N shows mRNA level of endocrine function-related gene visfatin, in normal diet, HFD-fed mice injected with JPH203, and control HFD-fed mice. Finally, Panel O shows mRNA level of osteoblastic factor-related gene runt-related transcription factor 2 (RUNX2) in normal diet, HFD-fed mice injected with JPH203, and control HFD-fed mice. [data shown as mean±SEM and significance was determined by one-way ANOVA; *P<0.05, ***P<0.001, ****P<0.0001.]

FIG. 8 shows the effect of JPH203 treatment on lipid storage and mitochondrial biogenesis in adipose tissues of HFD-fed mice, as described in Example 2. Panel A shows representative images of Nile red staining in epididymal and inguinal white adipose tissues (Zeiss confocal LSM800 20× lens), and Panels B and C shows quantitative analysis of epididymal white adipose tissue (Panel B) and inguinal white adipose tissue (Panel C) in normal diet, HFD-fed mice injected with JPH203, and control HFD-fed mice. Panel D shows representative images of Mitotracker staining in interscapular brown adipose tissues (Zeiss confocal LSM800 20× and 63× lens), and Panel E shows quantitative analysis of interscapular brown adipose tissue in normal diet, HFD-fed mice injected with JPH203, and control HFD-fed mice. Panel F shows representative images of Tom20 immuno staining in epididymal and inguinal white adipose tissues, and interscapular brown adipose tissues (Zeiss confocal LSM800 63× lens) in normal diet, HFD-fed mice injected with JPH203, and control HFD-fed mice. [“Epi fat”=epididymal white adipose tissue; “SubQ”=inguinal white adipose tissue; “iBAT”=interscapular brown adipose tissue; data shown as mean±standard error (SE) and significance was determined by one-way ANOVA or unpaired two-tailed t-test; *P<0.05, **P<0.01.]

FIG. 9 shows the effect of JPH203 treatment on glucose homeostasis in HFD-fed mice, as described in Example 2. Panel A shows fed blood glucose at day 6, Panel B shows fasting blood glucose at day 18, Panel C shows fed serum insulin at day 21, and Panel D shows fasting serum insulin before glucose administration during glucose tolerance test, in normal diet, HFD-fed mice injected with JPH203, and control HFD-fed mice. Panel E shows results of baseline intraperitoneal glucose tolerance testing (IPGTT), and Panel F shows area under the curve (AUC) of the testing results, in normal diet, HFD-fed mice injected with JPH203, and control HFD-fed mice. Panel G shows correlational analysis between weight and glucose levels in normal diet, HFD-fed mice injected with JPH203, and control HFD-fed mice; lines indicate significant correlations. [data shown as mean±SEM and significance was determined by one-way ANOVA (for analysis shown in Panels A, B, C, D, and G) or multiple unpaired t-test (for analysis shown in Panel F); *P<0.05, **P<0.01, ***P<0.001.]

FIG. 10 shows the effect of JPH203 treatment on adipogenesis in co-culture of differentiated adipocyte 3T3L1 and macrophage RAW 264.7 cells, as described in Example 3. Panel A shows a schematic diagram of the experimental design. Panel B shows mRNA expression adipogenic factor PPARy gene in fully differentiated adipocytes and preadipocytes in the presence of macrophages and treated with JPH203. Panel C shows representative images of ICC (Zeiss confocal LSM800 40× oil lens), and Panels D-G shows quantitative analysis. [Data shown as mean±SE and significance was determined by two-way ANOVA (for analysis shown in Panel B) and unpaired two-tailed t-test (for analysis shown in Panels D-G; *P<0.05, ***P<0.001, ****P<0.0001.]

### DETAILED DESCRIPTION OF THE INVENTION

The practice of the present invention will employ, unless otherwise indicated, conventional techniques of pharmaceutics, formulation science, protein chemistry, cell biology, and molecular biology, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

The terms “inhibit,” “reduce,” and “suppress” are used interchangeably and refer to any statistically significant decrease in occurrence or activity, including full blocking of the occurrence or activity. For example, “inhibition” can refer to a decrease of about 10%, 20%, 30%, 40%, 50%, 60%, 70%, 80%, 90% or 100% in activity or occurrence. An “inhibitor” is a molecule, factor, or substance that produces a statistically significant decrease in the occurrence or activity of a process, pathway, or molecule.

Terms such as “treating” or “treatment” or “to treat” refer to therapeutic measures that cure, slow down, lessen symptoms of, and/or halt progression of a diagnosed pathologic condition or disorder. In certain embodiments, a subject is successfully “treated” for a disease or disorder if the patient shows total, partial, or transient alleviation or elimination of at least one symptom or measurable physical parameter associated with the disease or disorder.

“Prevent” or “prevention” refers to prophylactic or preventative measures that prevent and/or slow the development of a targeted pathologic condition or disorder. Thus, those in need of prevention include those at risk of or susceptible to developing the disorder.

As used herein, “quantify” or “quantity” refers to the measurement of the amount of a component in a sample, and is typically calculated as a concentration such as a mass concentration (mass of the antibody divided by volume of the sample). Generally, quantity is determined in a sample that is collected from a subject.

An “effective amount” of an active agent is an amount sufficient to carry out a specifically stated purpose.

An “active agent” is an ingredient that is intended to furnish biological activity. The active agent can be in association with one or more other ingredients.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g., acetate, phosphate, or citrate buffer), a surfactant (e.g., polysorbate), a stabilizing agent (e.g., polyol or amino acid), a preservative (e.g., sodium benzoate), and/or other conventional solubilizing or dispersing agents.

“Large amino acid transporter small subunit1” or “LAT1” is encoded by the Slc7a5 gene and is a sodium-independent, high-affinity transporter of “large” neutral amino acids such as phenylalanine, tyrosine, leucine, arginine, and tryptophan. See UniProt Record Q01650 available on the world-wide web at uniprot. org/uniprot/Q01650, which is incorporated by reference. The LAT1 protein is expressed abundantly in “adult lung, liver, brain, skeletal muscle, placenta, bone marrow, testis, resting lymphocytes and monocytes, and in fetal liver.” UniProt Record Q01650. The LAT1 protein is also expressed to a much lesser extent in “thymus, cornea, retina, peripheral leukocytes, spleen, kidney, colon and lymph node.” UniProt Record Q01650.

“JPH203” refers to the compound(S)-2-Amino-3-(4-((5-amino-2- phenylbenzo[d]oxazol-7-yl)methoxy)-3,5-dichlorophenyl)propanoic acid, with a molecular formula of C₂₃H₁₉C₁₂N₃O₄. The JPH203 compound can also occur in the form of a hydrochloride salt. As used herein the term “JPH203” is understood to mean the compound or its pharmaceutical salt.

“OKY-034” refers to the compound (R)-5-phenyl-5-[4- (trifluoromethyl)phenoxy]pentane-1-amine fumarate, with a molecule formula C₁₈H₂₀F₃NO·C₄H₄O₄. The OKY-034 compound can also occur in the form of a salt. As used herein the term “OKY-034” is understood to mean the compound or its pharmaceutical salt.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

“Fat mass” is an indicator of the quantity of adipose tissue in a body or a portion of a body. Fat mass may be calculated as a weight, e.g., the weight of the adipose tissue in the body or a portion of the body; or may be calculated as a percentage, for example, a percentage of the weight of the body or portion of the body that constitutes adipose tissue. Methods of measuring fat mass as a percentage are known in the art, for example, by using skin calipers, body circumference measurements, body fat scales, waist circumference, dual-energy X-ray absorptiometry, hydrodensitometry, air displacement plethysmography, three-dimensional body scanner, etc.

“High-fat diet” or “HFD” refers to a diet in which the amount of diet in the diet is greater than recommended for a healthy diet, for example, greater than recommended by Food and Nutrition Board of the National Academies of Sciences Engineering, and Medicine, or by the U.S. Department of Health and Human Services and the U.S. Department of Agriculture. In some embodiments, a high-fat diet may be a diet in which at least 35% of total calories is consumed from fats, both unsaturated and saturated.

The term “metabolic syndrome” refers to a collection of conditions associated with metabolic disorder and increased risk of developing cardiovascular disease.

### Methods of the Invention

The present invention is directed to the use of a LAT1 inhibitor to treat obesity or otherwise reduce body weight or fat mass in a subject. As discussed in the Examples, Slc7a5 was surprisingly discovered to be upregulated in adipocytes in HFD-fed mice, and pharmacological inhibition of Slc7a5 by a LAT1 inhibitor unexpectedly prevented weight gain in these mice. In addition, the LAT1 inhibitor reduced leptin, reduced inflammation in adipose tissues, increased fatty acid beta oxidation, increased beiging/browning of white adipose tissues, increased mitochondrial biogenesis in brown adipose tissues, and improved glucose tolerance. These results demonstrate that LAT1 inhibition can reduce and/or prevent weight gain/obesity, and stop or slow the development of diet-associated metabolic syndrome.

Thus, the present invention is directed to methods of treating obesity in a subject in need thereof; methods of reducing body weight in a subject in need thereof; methods of inhibiting or preventing weight gain in a subject in need thereof; methods of reducing fat mass in a subject in need thereof; methods of preventing an increase in fat mass in a subject in need thereof; and methods of slowing the development of diet-associated metabolic disease in a subject in need thereof. These methods may comprise administering to the subject a pharmaceutical composition comprising an effective amount of a LAT1 inhibitor.

In some embodiments, the obesity, weight gain, or gain in fat mass may be diet-associated. In certain embodiments, the obesity, weight gain, or increase in fat mass may be associated with a high-fat diet.

In some embodiments, the reduction in fat mass or prevention of gain in fat mass may refer to the fat mass in the entire subject. In some embodiments, the reduction in fat mass or prevention of gain in fat mass may refer to the fat mass in a portion of the subject. Examples include, but are not limited to, interscapular brown adipose tissues, epididymal white adipose tissues, inguinal white adipose tissues, and combinations thereof.

In some embodiments, the methods of the invention may be used to slow the development of metabolic disorders associated with metabolic syndrome. These disorders include, but are not limited to, dyslipidemia, hypertension, impaired glucose tolerance, compensatory hyperinsulinemia, and the tendency to develop fat around the abdomen.

In some embodiments, the subject is a human, a non-human primate, a mouse, a rat, a dog or a cat. In preferred embodiments, the subject is a human.

Suitable dosage ranges of the LAT1 inhibitor, regardless of the route of administration, are generally about 0.0001 milligram to 2000 milligrams of the compound of the invention per kilogram body weight. In one specific embodiment, the dose is about 0.001 milligram to about 1500 milligrams per kilogram body weight, more specifically about 0.01 milligram to about 1000 milligrams per kilogram body weight, more specifically about 0.1 milligram to about 500 milligrams per kilogram body weight, and yet more specifically about 1 milligram to about 100 milligrams per kilogram body weight.

In carrying out the treatment methods described herein, any suitable method or route of administration can be used to deliver the active agents or combinations thereof described herein. The term “administration” includes any route of introducing or delivering the specified compositions or agents to subjects. In some embodiments the active agents or combinations thereof, are administered systemically. In some embodiments the active agents or combinations thereof, are administered locally. “Systemic administration” refers to introducing or delivering to a subject a specified composition or agent via a route which introduces or delivers the composition or agent to extensive areas of the subject's body (e.g., greater than 50% of the body), for example through entrance into the circulatory or lymph systems. By contrast, “local administration” refers to introducing or delivering to a subject a specified composition or agents via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body.

In some embodiments, administration can be carried out by any suitable route known in the art, including intratumoral, intravenous, subcutaneous, oral, topical, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. Administration includes self-administration and administration by another. The suitability of a given route or means of administration can be readily determined by a physician.

In some embodiments, the LAT1 inhibitor, or pharmaceutical compositions thereof, may be administered by, for example, by infusion or bolus injection, by absorption through epithelial or mucocutaneous linings (e.g., oral mucosa, rectal and intestinal mucosa, etc.), and may be administered together with another biologically active agent. Various delivery systems are known, e.g., encapsulation in liposomes, microparticles, microcapsules, capsules, etc., and can be used to administer a compound or composition of the invention.

In some embodiments, it may be desirable to administer one or more LAT1 inhibitors locally to the area in need of treatment, such as to adipose tissue. This may be achieved, for example, and not by way of limitation, by local infusion such as an epidural injection; topical application that can be absorbed through the skin or mucosal layers; by injection such as via a catheter; by a suppository; or by implant, with the implant being of a porous, non-porous or gelatinous material, including membranes, such as but not limited to silastic membranes, or fibers.

As used herein the term “effective amount” refers to an amount of an active agent as described herein that is sufficient to achieve, or contribute towards achieving, one or more of the outcomes listed in the “treatment” description herein. An appropriate “effective” amount in any individual case may be determined using standard techniques known in the art, such as dose escalation studies, and may be determined taking into account such factors as the desired route of administration (e.g., systemic vs. local), the desired frequency of dosing, etc. In some embodiments, the amount of a compound of the invention that will be effective in the methods described herein will depend on the nature or extent of the subject, and can be determined by standard clinical techniques. In addition, in vitro or in vivo assays may be employed to identify optimal dosage ranges. The precise dose to be employed in the formulations of the present invention will also depend on the route of administration and the extent of the condition, and dosing should be decided according to the judgment of the practitioner and each patient's circumstances.

For example, in some embodiments the dose of the LAT1 inhibitor may be calculated based on studies in humans or other mammals carried out to determine efficacy and/or effective amounts of the active agent. The dose amount and frequency or timing of administration may be determined by methods known in the art and may depend on factors such as pharmaceutical form of the active agent, route of administration, whether only one active agent is used or multiple active agents (for example, the dosage of a first active agent required may be lower when such agent is used in combination with a second active agent), and patient characteristics including age, body weight, or the presence of any medical conditions affecting drug metabolism.

In those embodiments described herein that refer to specific doses to be administered based on mouse studies, one of skill in the art can readily determine comparable doses for human studies based on the mouse doses, for example using the types of dosing studies and calculations described herein, using dose-response curves derived from in vitro or animal model test systems, etc.

In some embodiments suitable doses of the various active agents described herein can be determined by performing dosing studies of the type that are standard in the art, such as dose escalation studies, for example using the dosages shown to be effective in mice in the Examples section of this patent application as a starting point.

Dosing regimens can also be adjusted and optimized by performing studies of the type that are standard in the art, for example using the dosing regimens shown to be effective in mice in the Examples section of this patent application as a starting point. In some embodiments the active agents are administered daily, or twice per week, or weekly, or every two weeks, or monthly.

In some embodiments of the invention, suitable dosage ranges of the LAT1 inhibitor for parenteral, for example, intravenous administration can be 0.01 milligram to 100 milligrams per kilogram body weight, 0.1 milligram to 35 milligrams per kilogram body weight, and 1 milligram to 10 milligrams per kilogram body weight. Suitable dose ranges for oral delivery is about 0.01 milligram to about 100 milligrams of the LAT1 inhibitor per kilogram body weight, or from about 0.1 milligram to about 50 milligrams per kilogram body weight, or from about 0.5 milligram to about 20 milligrams per kilogram body weight, or from about 1 milligram to about 10 milligrams per kilogram body weight. Suitable dosage ranges of the LAT1 inhibitor for intranasal administration are generally about 0.01 pg/kg body weight to 1 mg/kg body weight. Suppositories generally contain 0.01 milligram to 50 milligrams of the LAT1 inhibitor per kilogram body weight. Suitable doses of the LAT1 inhibitor for topical administration are in the range of 0.001 milligram to 1 milligram, depending on the area to which LAT1 inhibitor is administered.

The present invention is also directed to methods of reducing expression of leptin in adipocytes and methods of reducing inflammation in adipose tissue. These methods comprise contacting adipocytes or adipose tissue with a LAT1 inhibitor or pharmaceutical composition thereof. In some embodiments, the methods may comprise direct administration of the LAT1 inhibitor or pharmaceutical composition thereof to the adipocytes or adipose tissue.

The efficacy of a given composition or method in treatment can be demonstrated or assessed using standard methods known in the art, such as methods that compare the efficacy of a given/“test” composition or method to a “control” composition or method. For example, the efficacy of a given composition or method in treating obesity or reducing weight or reducing fat mass may be demonstrated or assessed by comparing its ability to reduce weight or fat mass or improve one or more clinical indicators or symptoms of obesity as compared to that of a control composition or control method, such as a placebo control. For instance, a comparison can be made between different subjects (e.g., between a test group of subjects or a control group of subjects). Similarly, the efficacy of a given composition or method in treatment can be demonstrated or assessed in a single subject by comparing that subject's weight and/or clinical indicator(s) and/or symptom(s) before and after treatment.

In certain embodiments the compositions and methods provided herein may be employed together with other compositions and treatment methods known to be useful for treatment of obesity, reduction of body weight and/or fat mass, inhibition and/or prevention of weight gain, slowing of the development of diet-associated metabolic disease, etc., including, but not limited to, surgical methods (e.g., adjustable gastric banding, gastric bypass surgery, sleeve gastrectomy), pharmaceuticals (e.g., bupropion-naltrexone, liraglutide, orlistat, phentermine-topiramate), hydrogels, vagal nerve blockade, and the like, as well as other non-pharmaceutical and non-invasive methods known for initiating weight loss such as dietary changes and exercise/activity. Similarly, in certain embodiments the methods of treatment provided herein may be employed together with procedures used to monitor weight loss status/progression.

For example, in some embodiments the methods described herein and/or the agents and compositions described herein may be employed or administered to a subject prior to surgery, for instance, in order to reduce weight or fat mass if either is too high for gastric bypass surgery. In other embodiments the methods described herein and/or the agents and compositions described herein may be employed or administered to a subject both before and after performing surgery.

### LAT1 Inhibitors and Pharmaceutical Compositions Thereof

The LAT1 inhibitor may be any active agent that inhibits LAT1 activity. The LAT1 inhibitor may be, for example, a small molecule, nucleic acid (for instance, DNA, RNA, antisense RNA, interfering RNA, microRNA, catalytic RNA, catalytic DNA, etc.), peptide, or antibody or fragment thereof.

In some embodiments, the LAT1 inhibitor is a small molecule. In certain embodiments, the LAT1 inhibitor is selected from JPH203 and OKY-034. Other small molecule LAT1 inhibitors that may be used with the present invention are disclosed in U.S. Pat. Nos. 9,771,316 and 10,172,835 and in Napolitano et al. (2017), which are each incorporated herein by reference.

In some embodiments, the LAT1 inhibitor is a nucleic acid. Examples of nucleic acids that can be used to inhibit LAT1 include short interfering nucleic acids disclosed in U.S. Pat. No. 10,590,422, which are each incorporated herein by reference.

In some embodiments, the LAT1 inhibitor may be modified to promote the LAT1 inhibitor to target or enter adipose tissue or adipocytes. The LAT1 inhibitor may be conjugated or otherwise linked to a ligand such as a small molecule, aptamer, peptide, or antibody that targets adipose tissue or adipocytes. Examples include, but are not limited to, the DNA aptamer Adipo8, MA-33, and 91 (see, e.g., Kim et al., 2014; which is incorporated herein by reference).

In some embodiments, the LAT1 inhibitor may be modified to target antigens that are differentially expressed on adipocytes. Such antigens include, but are not limited to, AD3 and tyrosine kinase-like orphan receptor 1 (“ROR1”).

In embodiments in which the LAT1 inhibitor is a nucleic acid, the LAT1 inhibitor may be incorporated into a vector that can transduce adipose tissue. For example, the vector may be an adeno-associated viral (AAV) vector as described in U.S. Pat. No. 10,711,281, which is incorporated herein by reference (see also Carlotti et al., 2004; which is incorporated herein by reference).

In some embodiments, the LAT1 inhibitor may be formulated in a pharmaceutical composition. The pharmaceutical composition may comprise one or more carriers, diluents, excipients, or other additives. Carriers can be liquids, such as water and oils, including those of petroleum, animal, vegetable or synthetic origin, such as peanut oil, soybean oil, mineral oil, sesame oil, and the like. The carriers can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the invention and pharmaceutically acceptable vehicles are preferably sterile. Sterile water can be a vehicle when the compound of the invention is administered intravenously. Saline solutions and aqueous dextrose and glycerol solutions can also be employed as liquid vehicles, particularly for injectable solutions.

As further examples, the composition can comprise one or more stabilizing agents (e.g., dextran 40, glycine, lactose, mannitol, trehalose, maltose), one or more buffers (e.g., acetate, citrate, histidine, lactate, phosphate, Tris), one or more pH adjusting agents (e.g., hydrochloric acid, nitric acid, potassium hydroxide, sodium hydroxide), one or more surfactants (polysorbate, sodium lauryl sulfate, polyethylene glycol-fatty acid esters, lecithins), and/or one or more diluents (e.g., water, physiological saline). The composition may also include excipients such as starch, glucose, lactose, methyl cellulose, sucrose, gelatin, malt, rice, flour, chalk, silica gel, sodium stearate, glycerol monostearate, talc, sodium chloride, dried skim milk, glycerol, propylene, glycol, water, ethanol, and the like. The present compositions, if desired, can also contain minor amounts of wetting or emulsifying agents, or pH buffering agents, or solubilizers (e.g., sulfobutylether b-cyclodextrin). The pH of the composition is preferably between about 3.0 and 8.0. In certain embodiments, the pH is between about 4.0 and 7.0, or between about 5.0 and 6.5.

The present formulations can take the form of solutions, suspensions, emulsion, tablets, pills, pellets, capsules, capsules containing liquids, powders, sustained-release formulations, suppositories, emulsions, aerosols, sprays, suspensions, or any other form suitable for use.

In some embodiments, the compositions of the invention are formulated in accordance with routine procedures as a pharmaceutical composition adapted for intravenous administration to subjects. Typically, pharmaceutical compositions of the invention for intravenous administration are solutions in sterile isotonic aqueous buffer. Where necessary, the pharmaceutical compositions may also include a solubilizing agent. Pharmaceutical compositions for intravenous administration may optionally include a local anesthetic such as lignocaine to ease pain at the site of the injection. Generally, the ingredients of the pharmaceutical compositions of the present invention are supplied either separately or mixed together in unit dosage form, for example, as a dry lyophilized powder or water free concentrate in a hermetically sealed container such as an ampoule or sachet indicating the quantity of active agent. Where the LAT1 inhibitor of the invention is to be administered by infusion, it can be dispensed, for example, with an infusion bottle containing sterile pharmaceutical grade water or saline. Where the LAT1 inhibitor of the invention is administered by injection, an ampoule of sterile water for injection or saline can be provided so that the ingredients may be mixed prior to administration.

In some embodiments, the pharmaceutical compositions of the invention can be administered orally. Formulations for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the pharmaceutical compositions may be coated to delay disintegration and absorption in the gastrointestinal tract thereby providing a sustained action over an extended period of time. Selectively permeable membranes surrounding an osmotically active driving compound are also suitable for orally administered compounds of the invention. In some platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc.

The pharmaceutical composition of the invention may be prepared by methods known in the art. For example, the methods may comprise admixing a LAT1 inhibitor and a pharmaceutically acceptable carrier to prepare the composition.

An aspect of the present invention relates to the LAT1 inhibitor, or pharmaceutical composition thereof, as described herein, for use in any of the methods of the present invention described herein.

The invention further provides pharmaceutical packs or kits comprising one or more containers filled with the LAT1 inhibitor or pharmaceutical composition thereof. Optionally associated with such container(s) can be a notice in the form prescribed by a governmental agency regulating the manufacture, use or sale of pharmaceuticals or biological products, which notice reflects approval by the agency of manufacture, use or sale for human administration.

The invention is further described in the following non-limiting Examples, as well as the Figures referred to therein.

### EXAMPLES

### Example 1

Expression of Slc7a5 was examined in adipose tissues of mice after being fed with either a high fat diet (HFD) containing 45% kcal fat, or normal control diet containing 10% kcal fat (n=6 per group) for 3 months.

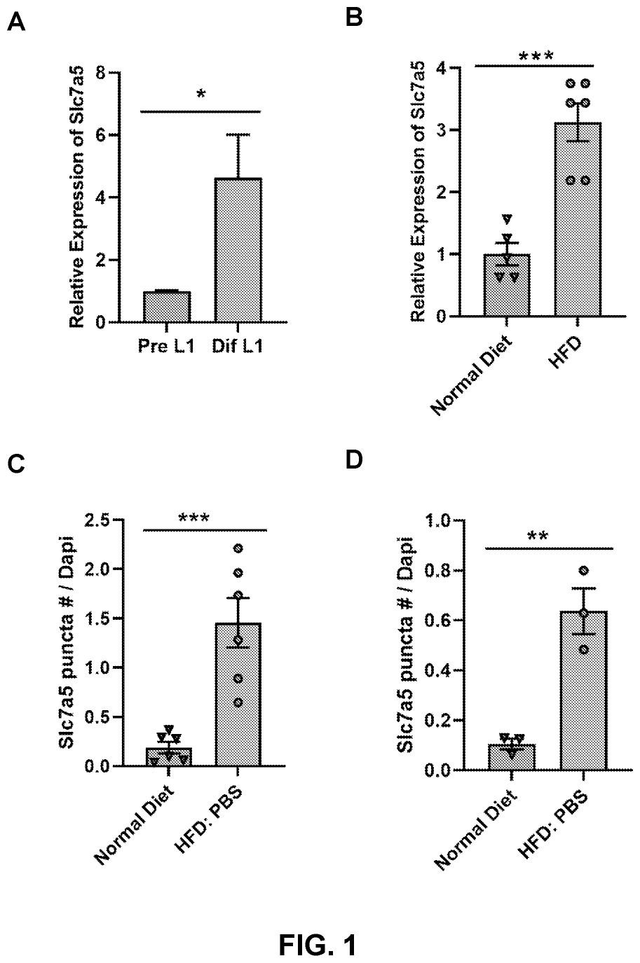

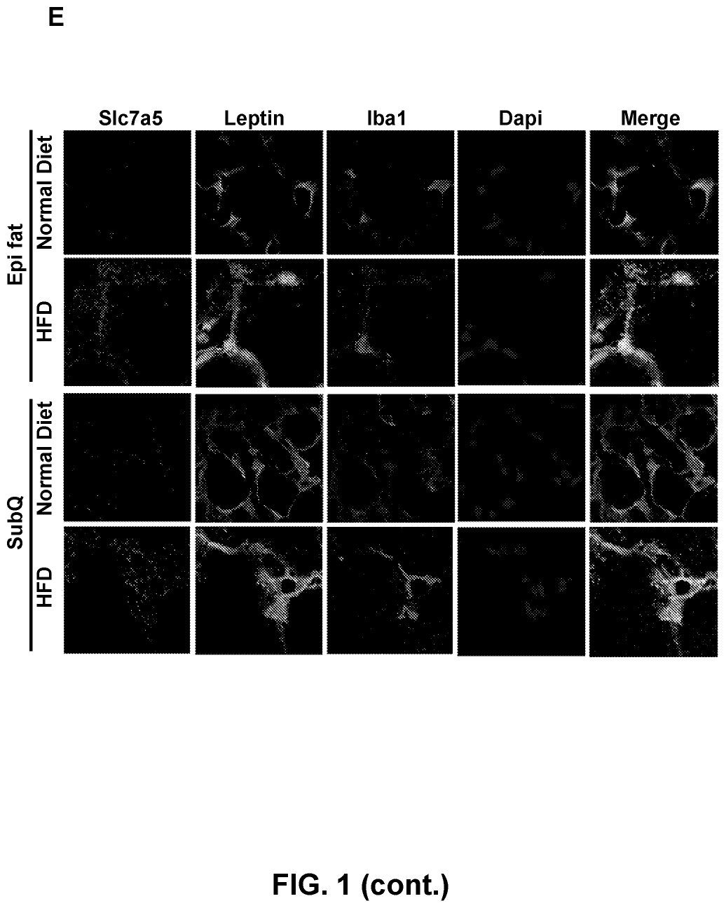

The results showed that Slc7a5 is upregulated in fully differentiated adipocytes (see FIG. 1, Panel A) and in adipose fat tissues (see FIG. 1,l Panels B-D) of HFD-fed mice as compared to the normal-diet mice. The increased expression of Slc7a5 in the HFD-fed mice was evident in both epididymal and inguinal fat tissues (see FIG. 1, Panels C-E).

These results suggest that amino acid transport may fuel the metabolic response of adipocytes to excess leptin intake.

### Example 2

A study was conducted to determine if inhibiting Slc7a5 prevents the metabolic response of adipocyte to excess leptin intake and obesity. Mice fed with HFD for 3 months were then injected intraperitoneally with either (a) JPH203, a potent Slc7a5 inhibitor, at a dose of 25 mg/kg, or (b) PBS (vehicle) as control, for 15 consecutive days while on HFD (Days 1-15). Mice fed with a normal diet and receiving no injection were included as a comparator.

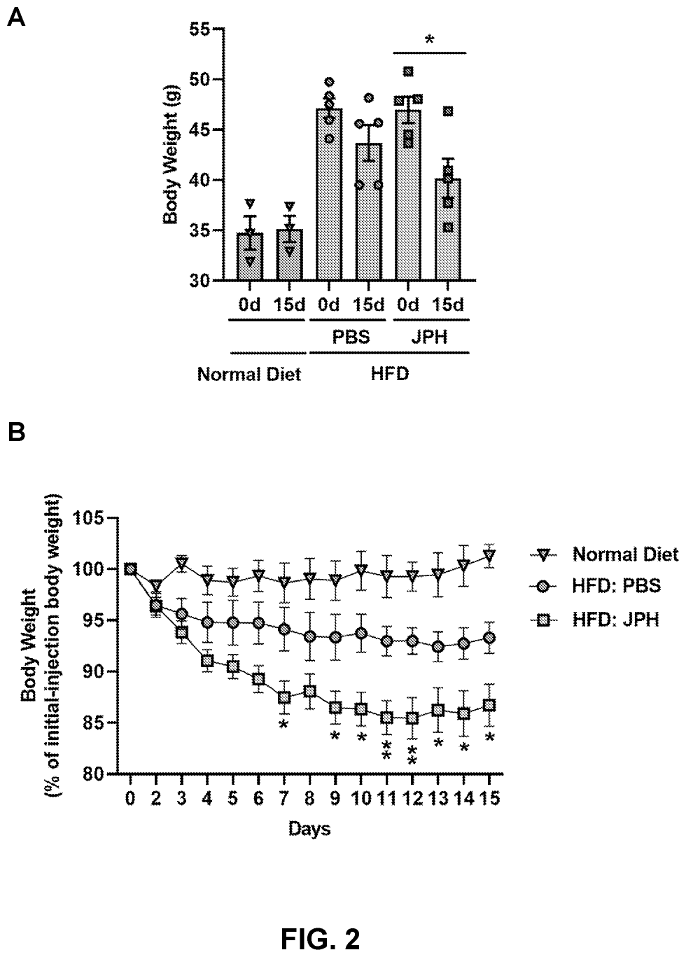

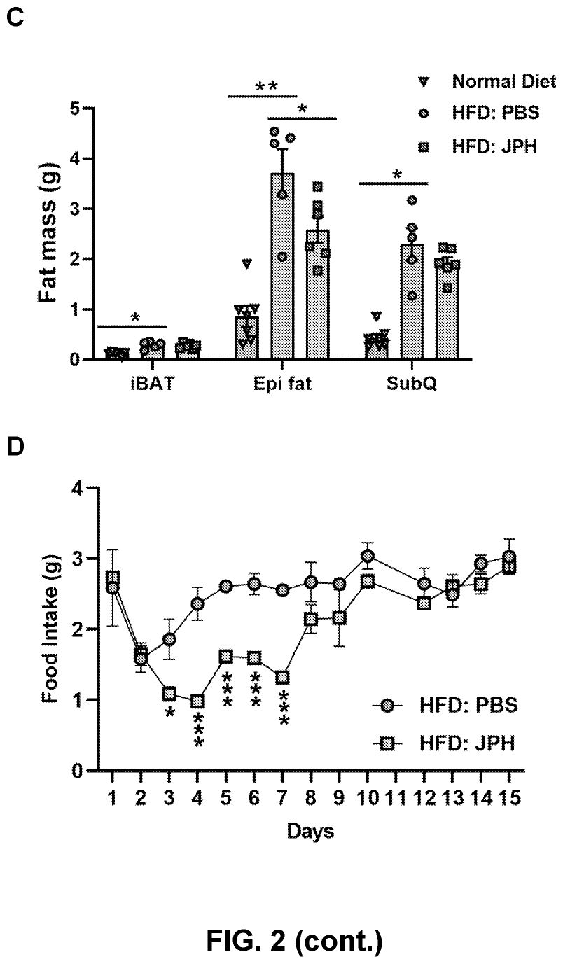

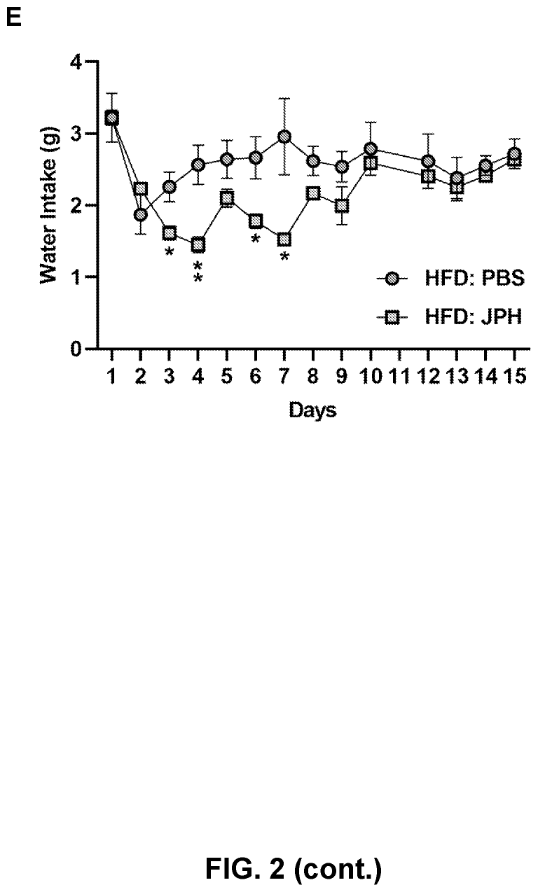

The results show that HFD-fed mice injected with the vehicle nearly doubled in weight as compared to mice on normal diet (see FIG. 2, Panel A). However, HFD-fed mice injected with JPH203 treated mice displayed significant weight reduction after 15 days (see FIG. 2, Panels A and B). At 15 days, the mass of epididymal white adipose tissue was significantly less in the HFD-fed mice injected with JPH203 as compared to the control HFD-fed mice (see FIG. 2, Panel C). In addition, at 15 days, mass of interscapular brown adipose tissue and epididymal and inguinal white adipose tissue was significantly less in the normal fed mice as compared to the control HFD-fed mice, but not significantly less in the normal fed mice as compared to the HFD-fed mice injected with JPH203 (see FIG. 2, Panel C). Notably, appetite suppression was not a major contributing factor to the weight loss that occurred in the HFD-fed mice injected with JPH203 (see FIG. 2, Panels D and E).

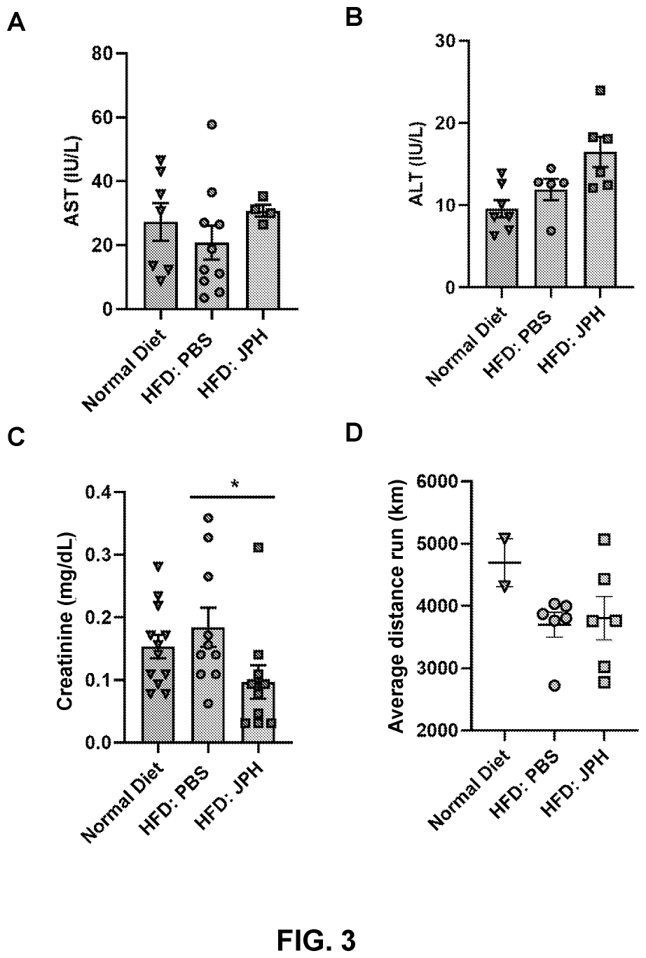

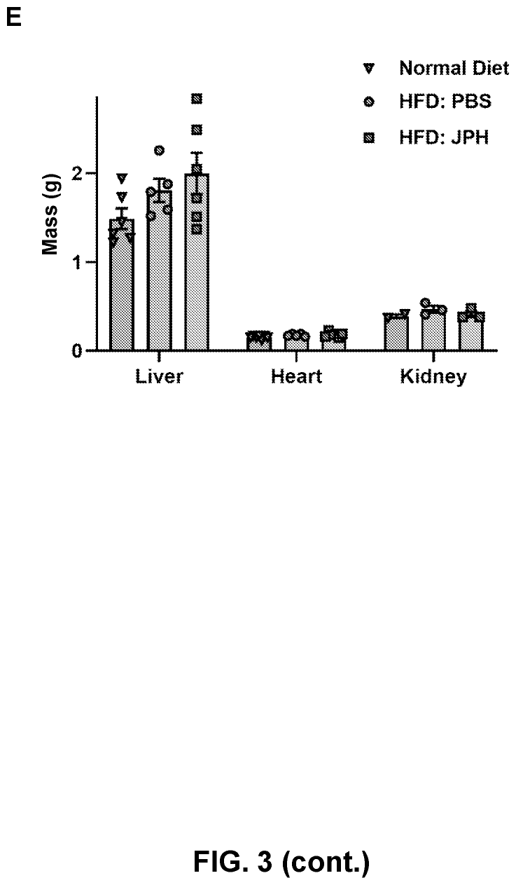

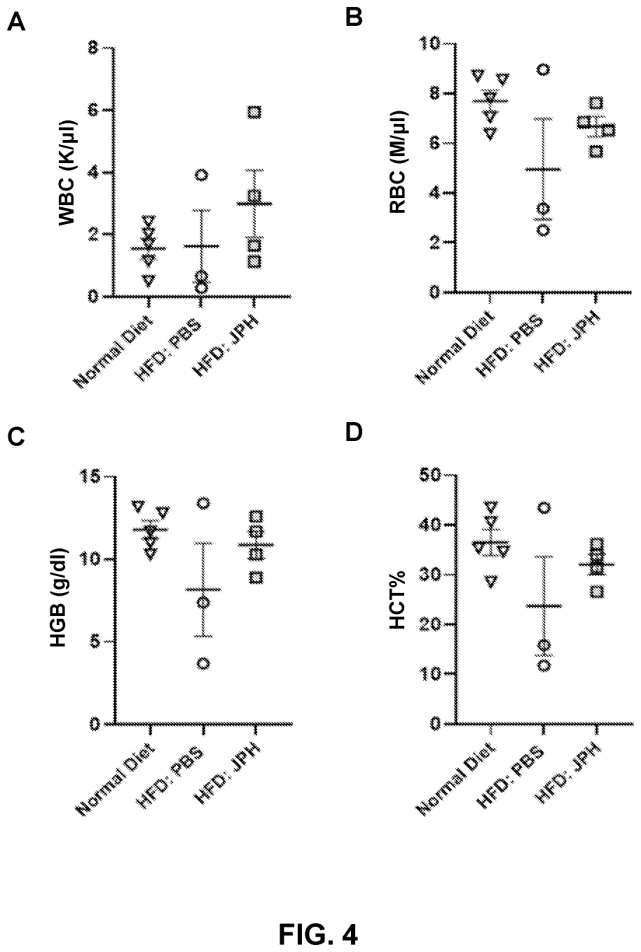

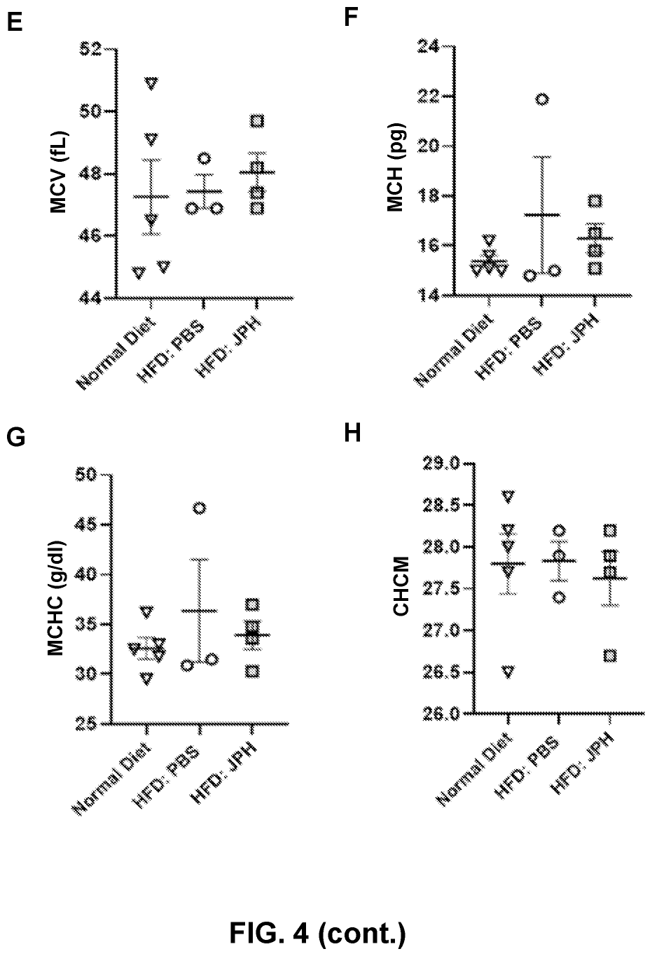

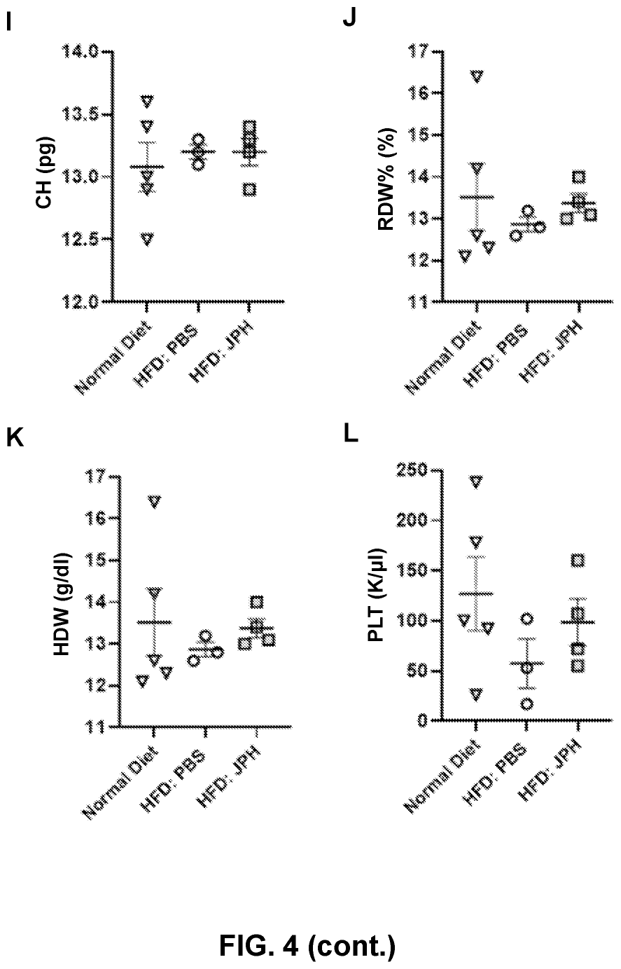

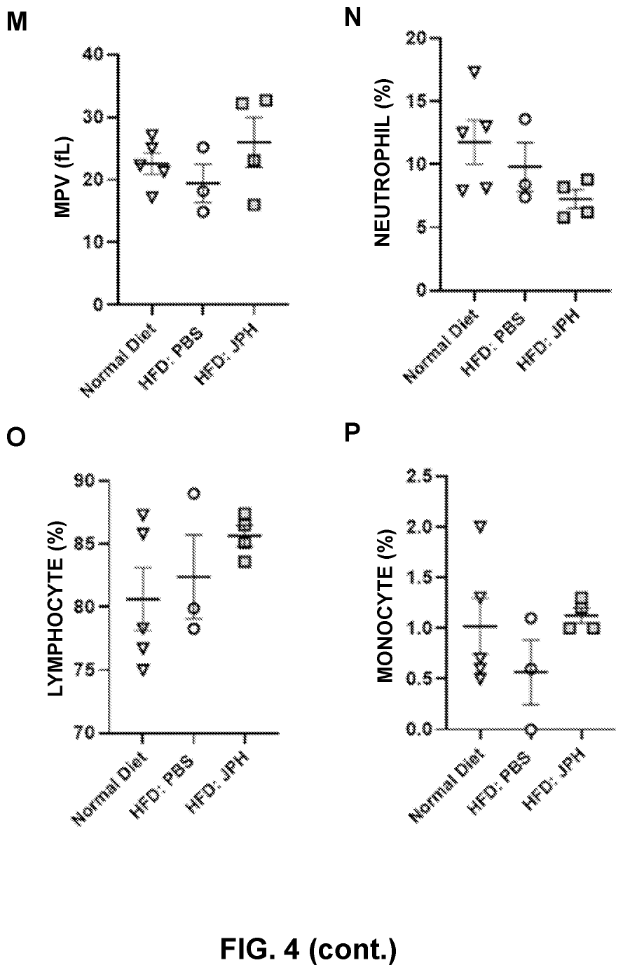

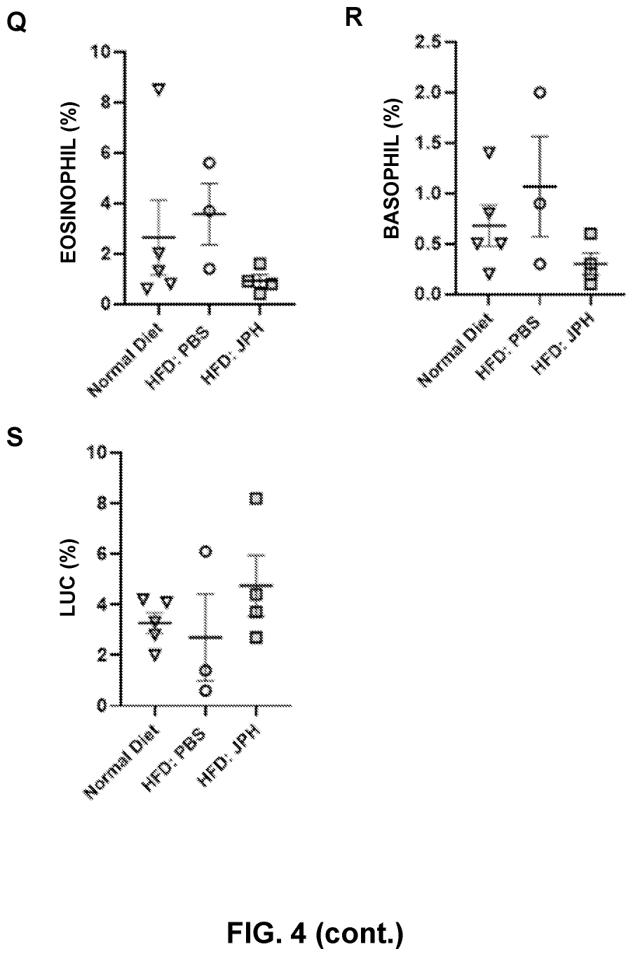

In addition, liver function (AST, ALT), kidney function (creatinine), and effect on bone marrow and immune response (whole blood counts) were tested to examine the toxicity of the pharmacological JPH203 treatment. HFD-fed mice treated with JPH203 exhibited AST and ALT serum levels in the normal range (normal is 13-35 IU/L and 37-88 IU/L, respectively) (see FIG. 3, Panels A and B). Creatine levels in the HFD-fed, JPH203-treated mice were also in the normal ranges, and were significantly less than the creatine levels of the control HFD-fed mice (see FIG. 3, Panel C). A running wheel experiment showed that JPH203 treatment does not decrease mice activity (see FIG. 3, Panel D). Also, there was no effect of JPH203 treatment on liver, heart, or kidney weight (see FIG. 3, Panel E). Further, the complete blood counts (CBC), which can evaluate overall health of mice and detect a variety of diseases and conditions, showed no significant effect on bone marrow and immune response after JPH203 treatment (see FIG. 4, Panels A-S).

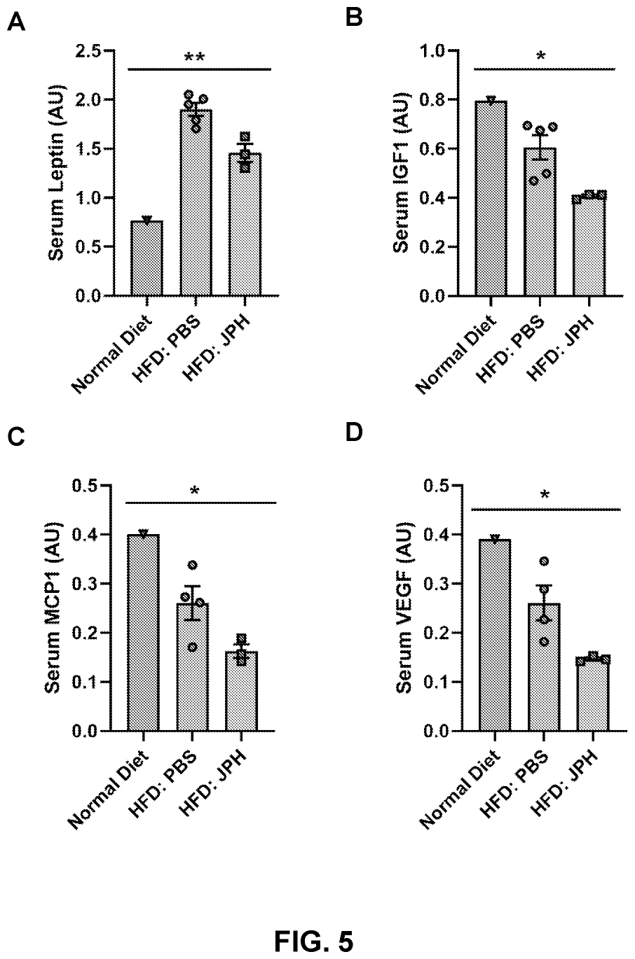

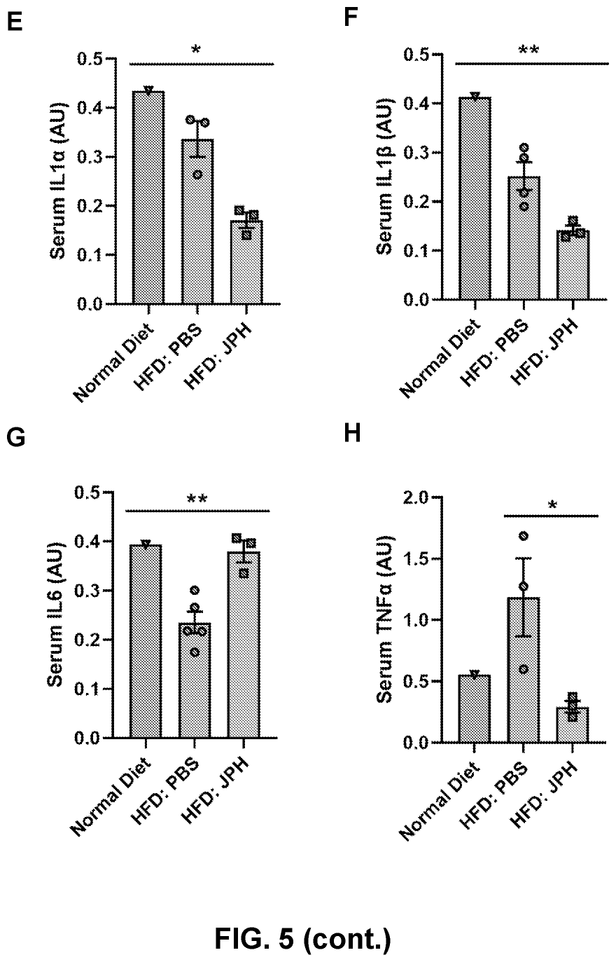

Obesity is correlated with chronically high levels of several cytokines which produced primarily by adipose tissues, which play a role in chronic inflammatory states. Compared to control HDF-fed mice, HFD-fed mice injected with JPH203 exhibited decreased serum levels of leptin, TNFα, IGF1, VEGF, MCP1, IL1α, IL1β, but increased serum level of IL6 (see FIG. 5, Panels A-H).

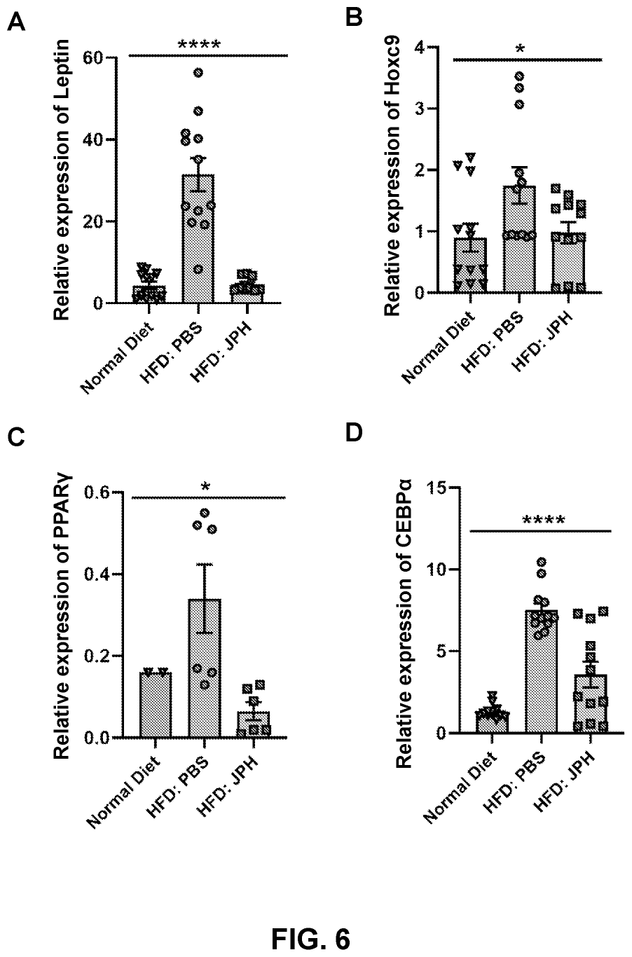

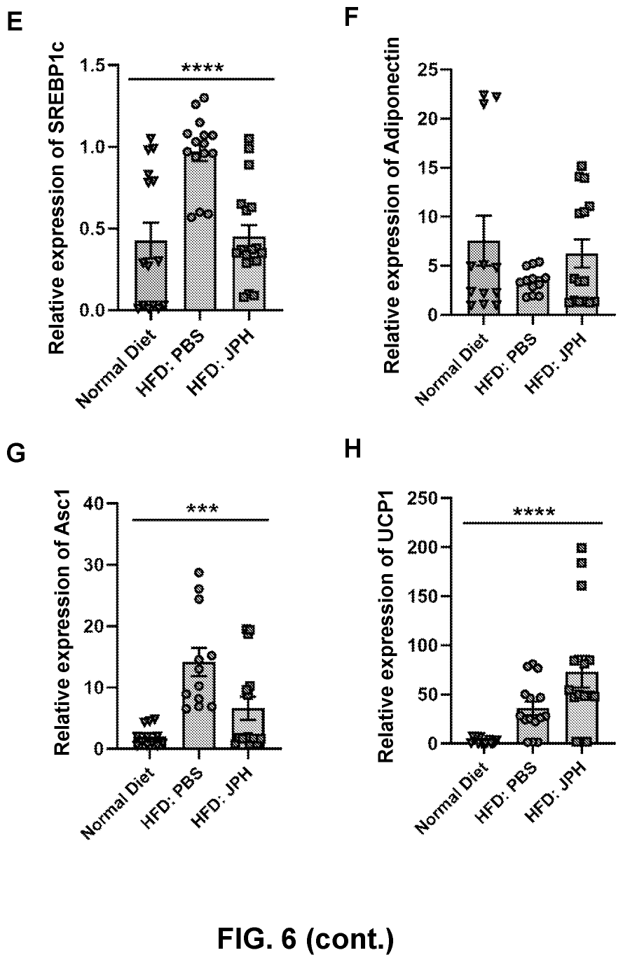

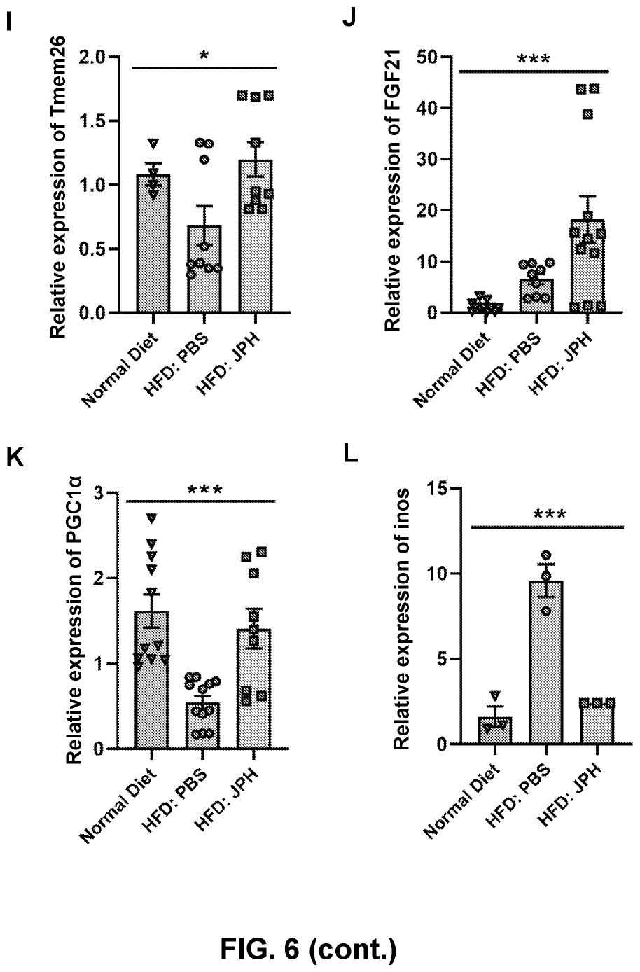

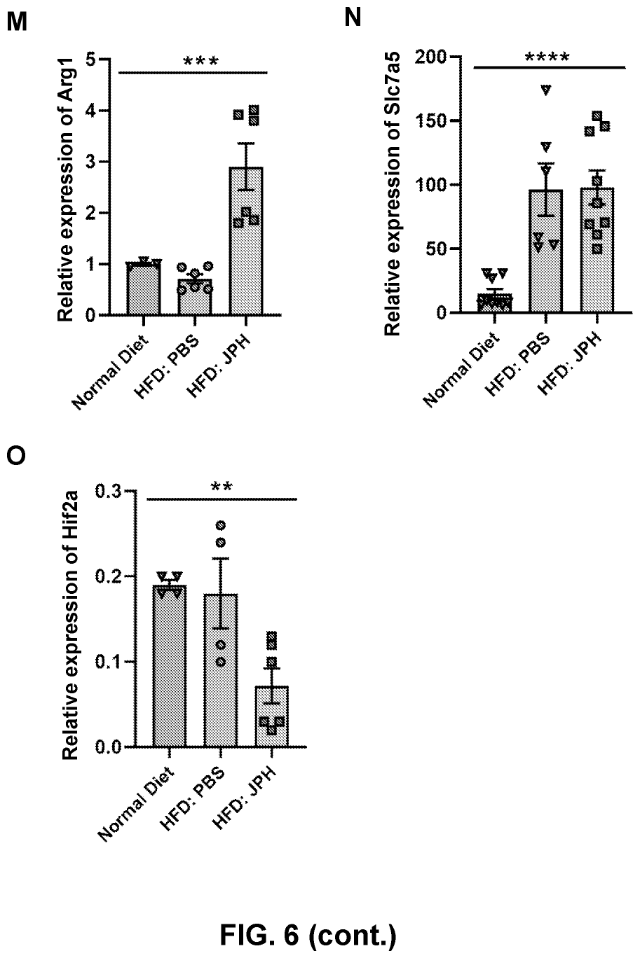

To determine if JPH203 affected adipose tissue function, the epididymal and inguinal white adipose tissues were dissected and analyzed 15 days after treatment. An analysis of the adipocyte gene expression profiles in the epididymal white adipose tissue indicated that JPH203 treatment increased the mRNA levels for beiging and browning markers (see FIG. l , Panels H-K) and decreased mRNA levels for general adipogenic markers (see FIG. 6, Panels A-E) and white adipogenic markers (see FIG. 6, Panel G) as compared to the control HFD-fed mice. JPH203 also reduced proinflammatory nitric oxide synthase 2 (Nos2/Inos) gene expression and increased anti-inflammatory arginase 1 (Arg1) gene expression (see FIG. 6, Panels L and M, respectively). JPH203 treatment did not change Slc7a5 expression, but physically blocked the function of LAT1 as a neutral amino acid transporter, as indicated by the decreased Hif2α expression, which is the downstream target of mTOR signaling (see FIG. 6, Panels N and O).

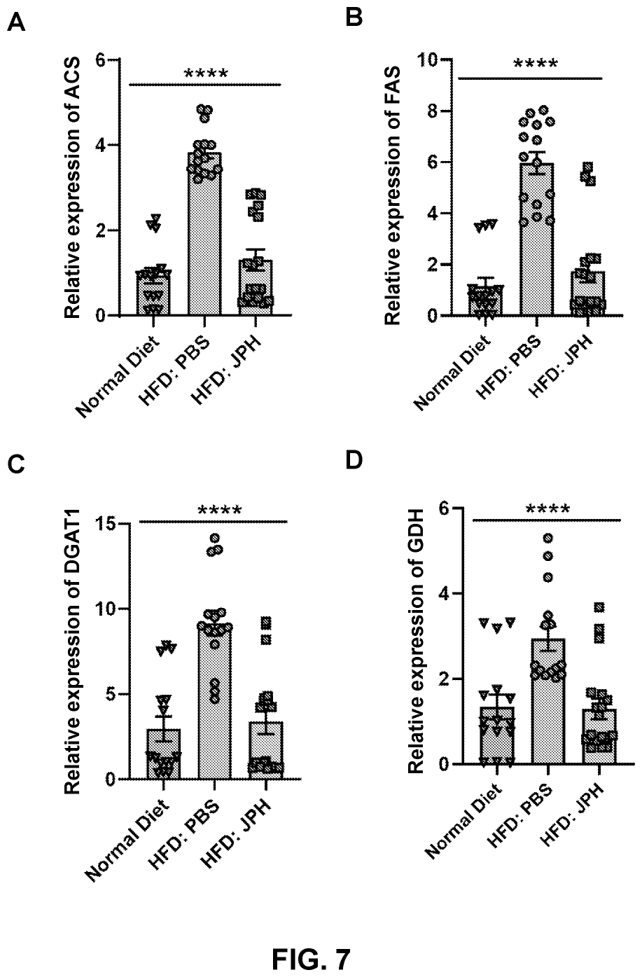

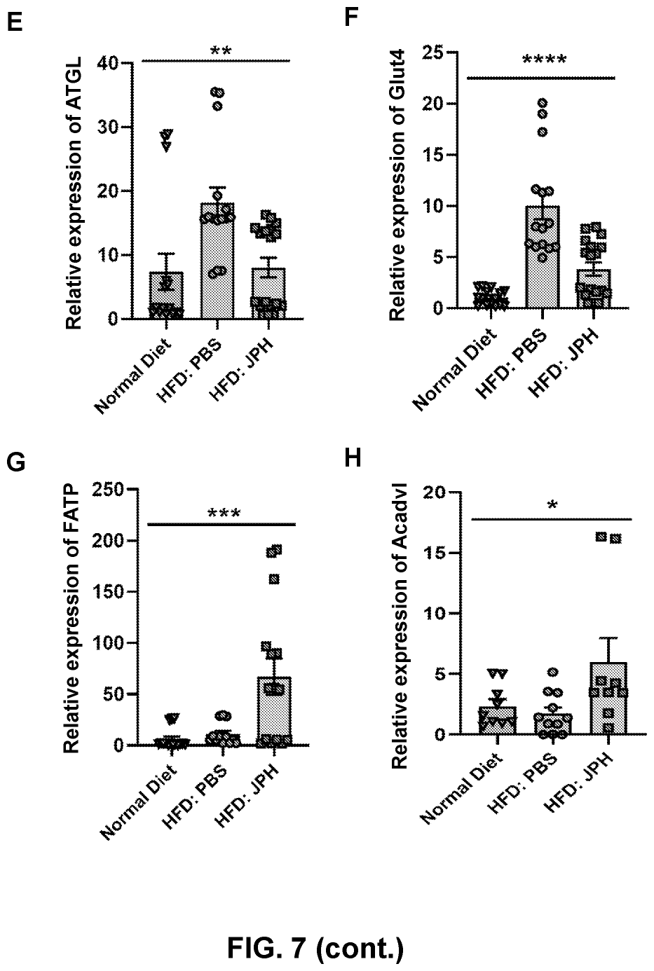

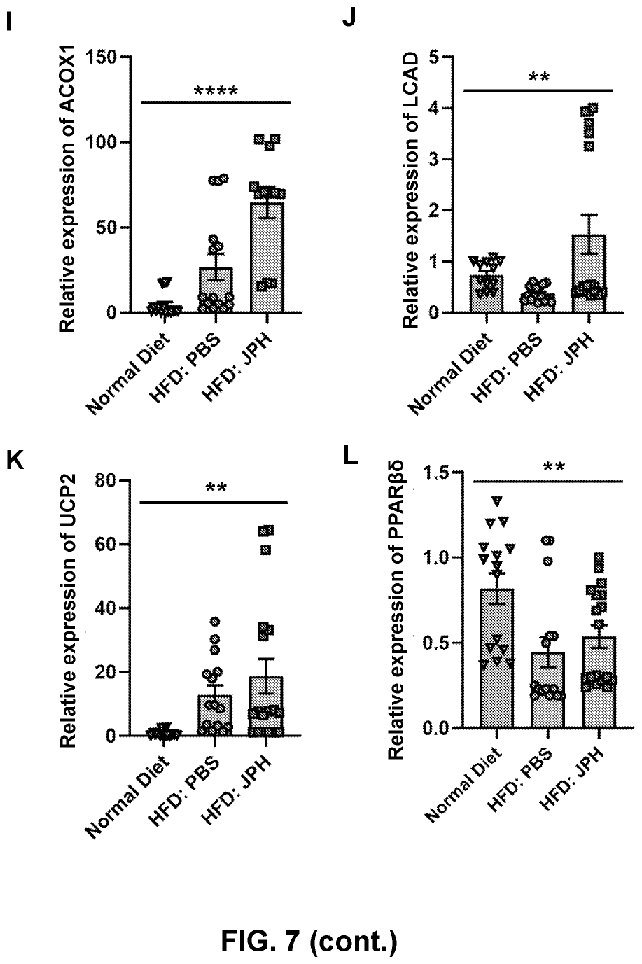

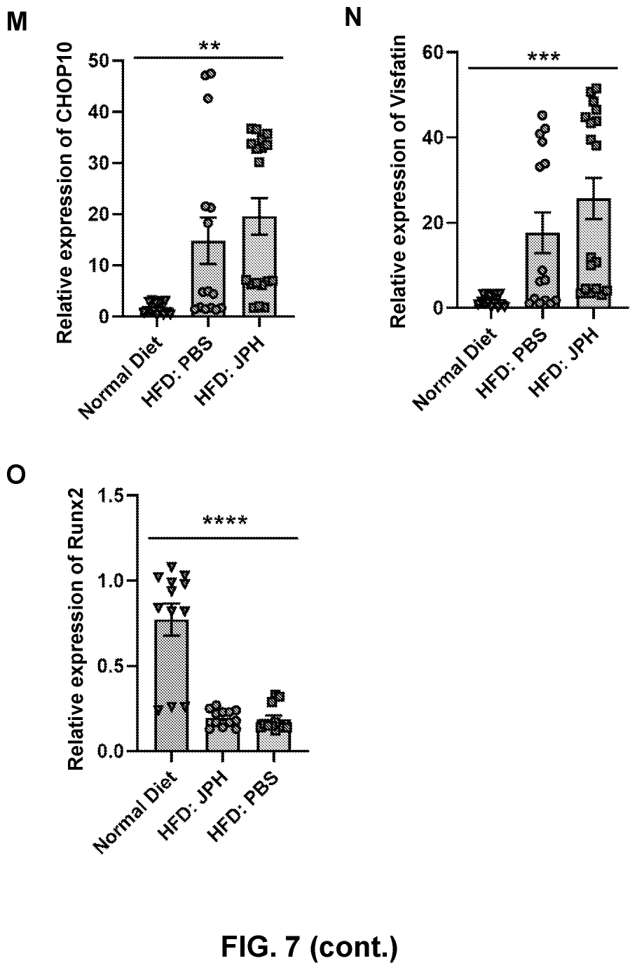

Administration of JPH203 in HFD-fed mice also showed an effect on lipid homeostasis in epididymal adipose tissues. Genes related to glyceroneogenesis/fatty acid synthesis, lipogenesis, pentose phosphate pathway (NADPH production), lipolysis, and glucose uptake/glycolysis were downregulated in HFD-fed mice injected with JPH203 as compared to control HFD-fed mice (see FIG. 7, Panels A-F). Moreover, genes related to fatty acid uptake, energy dissipation/fatty acid β oxidation, and anti-adipogenic factor were upregulated in HFD-fed mice injected with JPH203 as compared to control HFD-fed mice (see FIG. 7, Panels G-L, M).

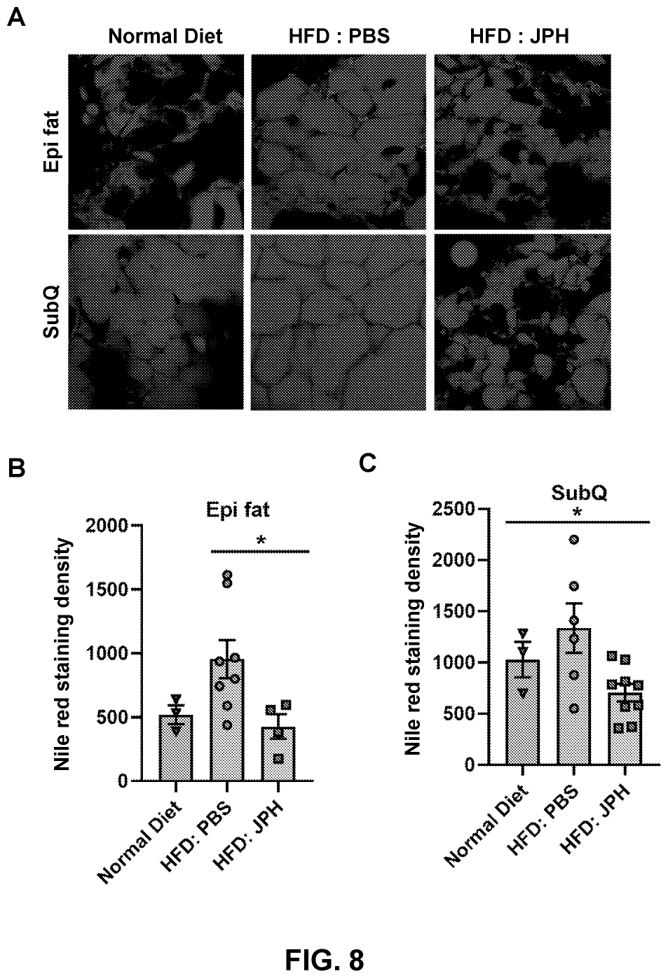

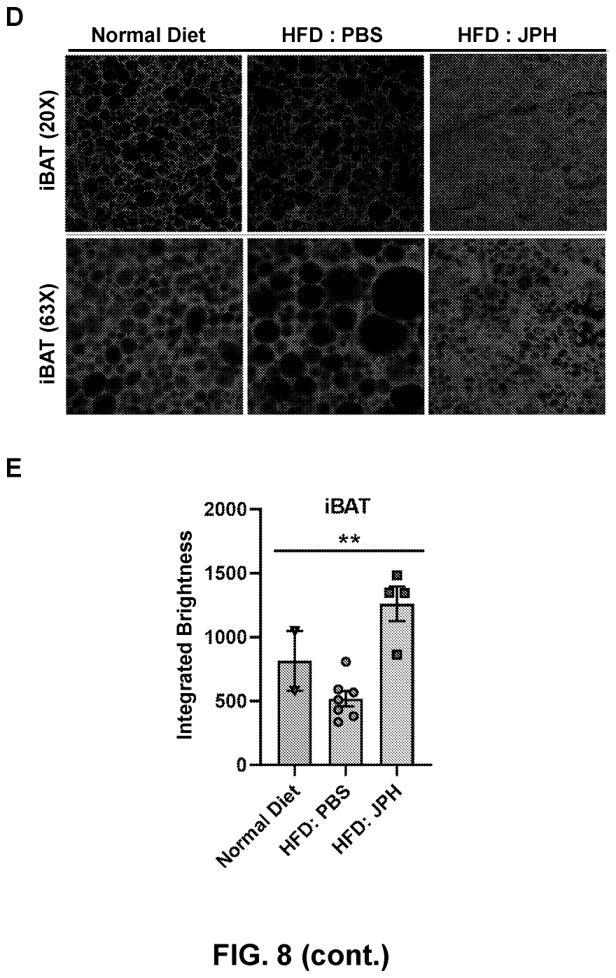

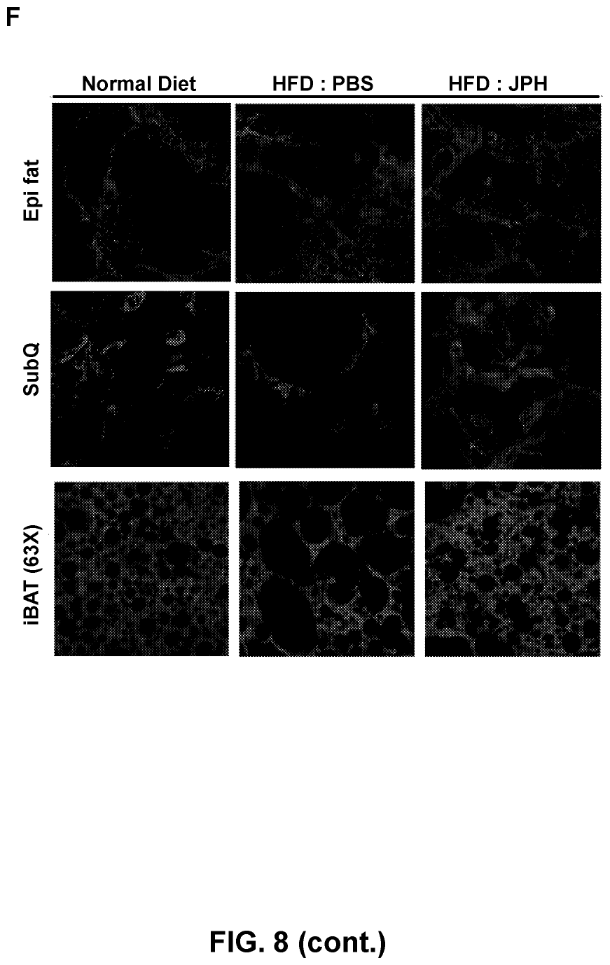

Nile red staining showed that that adipocytes from control HFD-fed mice displayed a hypertrophic, lipid-laden morphology, whereas HFD-fed mice injected with JPH203 displayed smaller sized adipocytes with lower lipid content, similarly to that observed in mice under normal diet (see FIG. 8, Panels A-D). Also, JPH203 mediated the induction of morphological changes and significantly increased the mitochondrial biogenesis in interscapular brown adipose tissues (see FIG. 8, Panels D and E). The effect of a double dose of JPH203 as compared to a single dose of JPH203 is exhibited in FIG. 8, Panel F.

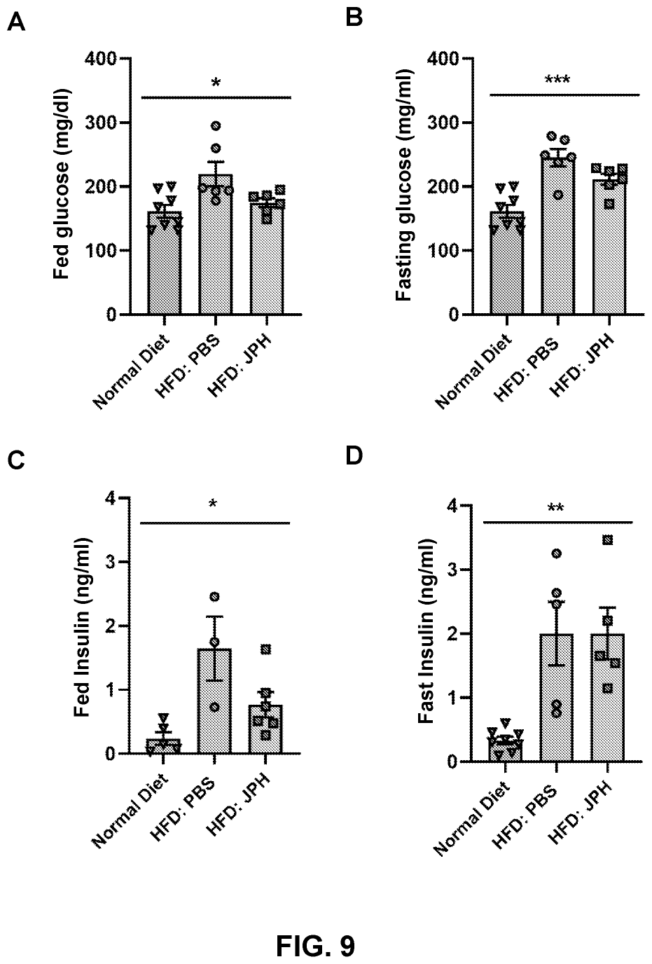

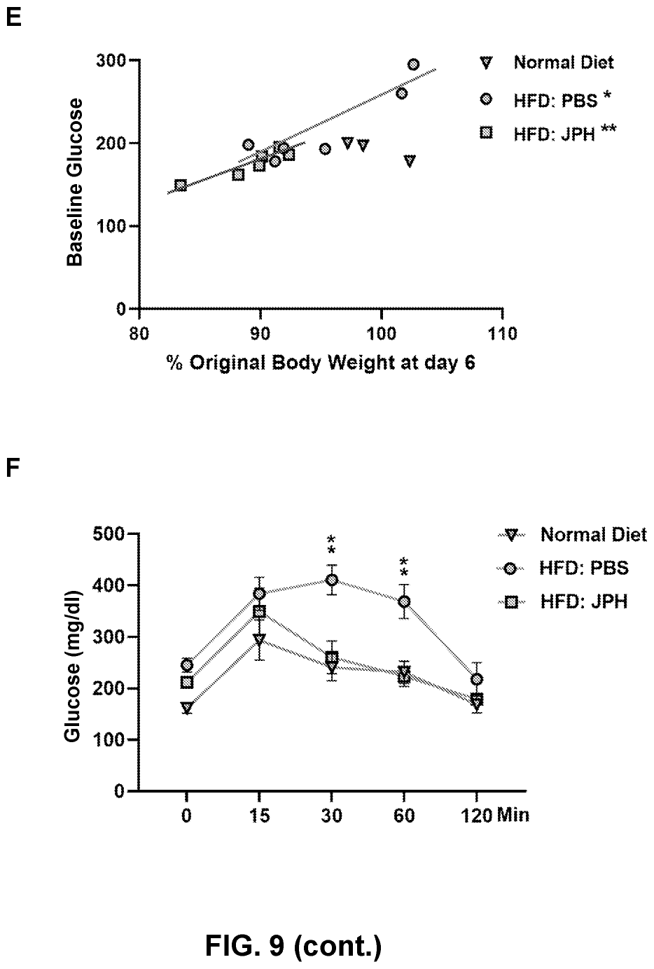

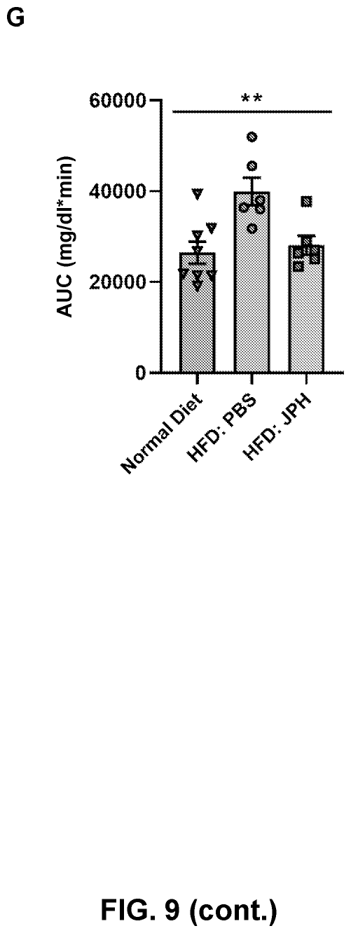

Mice that were obese due to HFD were hyperglycemic and hyperinsulinemia regardless of fed or fasting status (see FIG. 9, Panels A-D). However, HFD-fed mice injected with JPH203 exhibited lower fed and fasting glucose and improved glucose tolerance as compared to the control HFD-fed mice (see FIG. 9, Panels A-D). HFD-fed mice injected with JPH203 also exhibited significantly faster reduction in blood glucose concentration after glucose administration (see FIG. 9, Panel E) and AUC (glucose area under the curve) index (see FIG. 9, Panel F). Furthermore, weight loss in HFD-fed mice injected with JPH203 was positively correlated with decreased baseline glucose when compared with control HFD-fed mice (see FIG. 9, Panel G).

Together, these results suggest that inhibiting Slc7a5 decreased the lipid content in white adipose tissue, and increased white adipocyte “browning” by promoting energy dissipation and fatty acid breakdown.

### Example 3

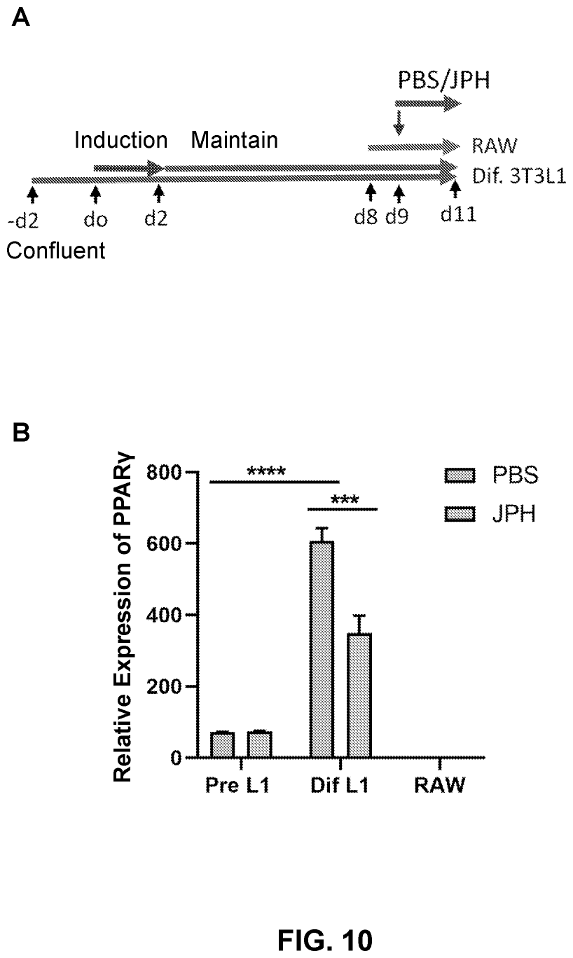

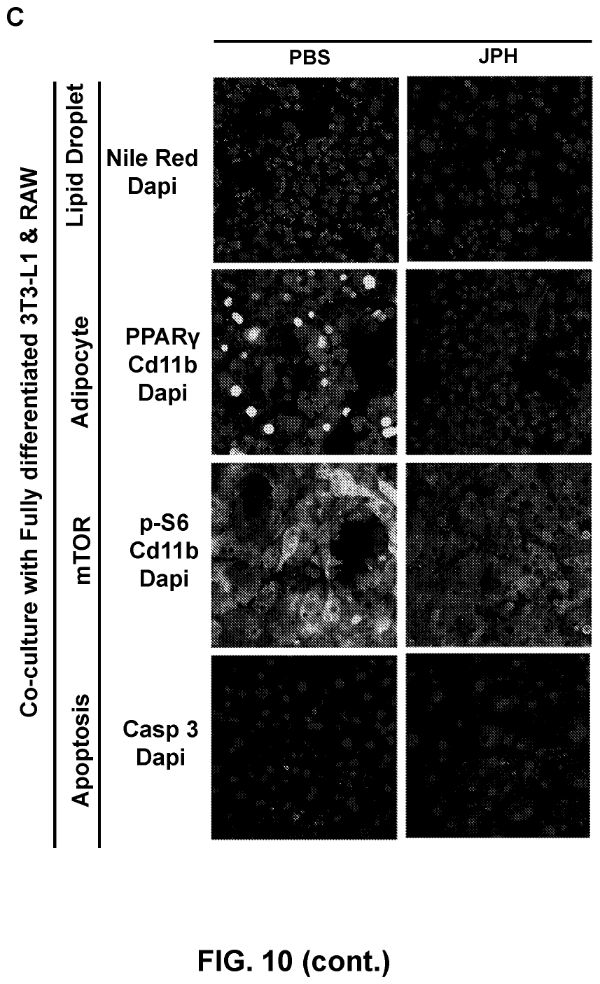

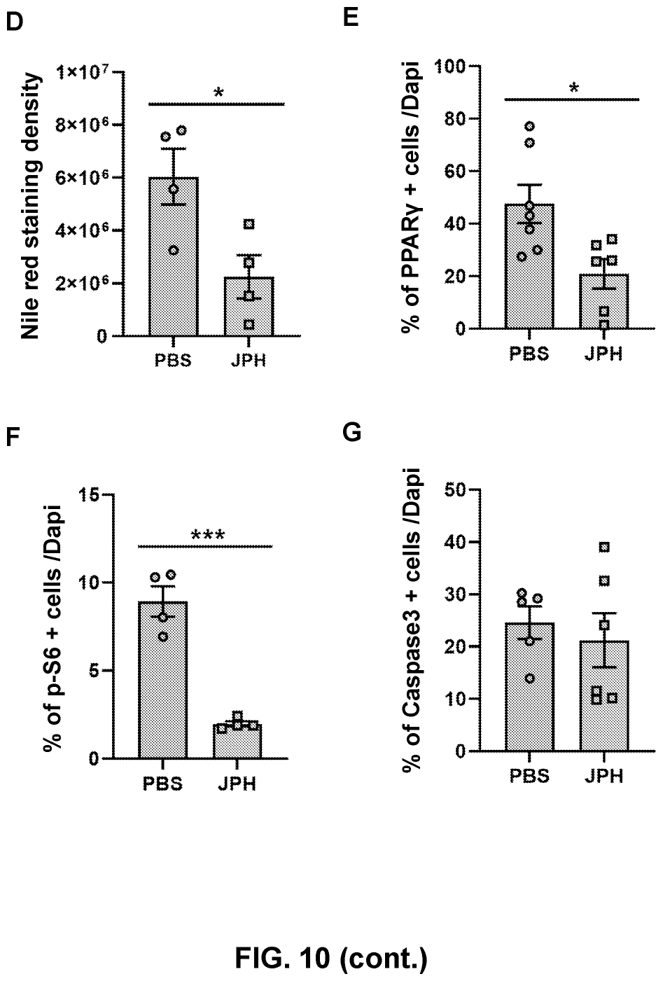

A study was conducted to determine which aspects of the adipocyte lineage was affected by inhibition of Slc7a5. To this end, pre-adipocytes or differentiated adipocytes 3T3L1 were co-cultured in the presence of macrophage (RAW 264.7 cells). The experimental design is illustrated in FIG. 10, Panel A.

The study examined expression of PPARγ and mTOR, and Nile red stained lipid droplets, which are indicators of adipogenesis and are shown to increase in differentiated adipocytes compared to preadipocytes. The results show that Slc7a5 inhibition by JPH203 treatment reduced PPARγ and mTOR expression in the fully differentiated adipocytes as compared to the preadipocytes (see FIG. 10, Panel B). Further, JPH203treatment decreased adipocyte differentiation by reduction of lipid droplets and PPARγ through mTOR signaling without causing cell death (see FIG. 10, Panels C-G), revealing that JPH203 does not affect cell survival.

Together, these results show that inhibiting Slc7a5 in differentiated adipocytes decreases adipogenesis.

### REFERENCES

Ades P. A. & Savage P. D., Obesity in coronary heart disease: an unaddressed behavioral risk factor, Preventive Medicine, 104:117-119 (2017).
Carlotti F., et al., Lentiviral vectors efficiently transduce quiescent mature 3T3-LI antibodies, Molecular Therapy, 9(2): 209-217 (2004).
Church C. D., et al., WAT is a functional adipocyte, Adipocyte, 1(1): 38-45 (2012).
Grundy S. M., et al. Diagnosis and management of the metabolic syndrome: an American Heart Association/National Heart, Lung, and Blood Institute Scientific Statement, Circulation, 112(17): 2735-2752 (2005).
Kim E. Y., et al., Selection of aptamers for mature white adipocytes by cell SELEX using flow cytometry, PLOS One, 9(5): e99747 (2014).
Napolitano L., et al., Potent inhibitors of human LAT1 (SLC7A5) transporter based on dithiazole and dithiazine compounds for development of anticancer drugs, Biochemical Pharmacology, 143:39-52 (2017).
Roberts D. L., et al., Biological mechanisms linking obesity and cancer risk: new perspectives, Annual Review of Medicine, 61:301-316 (2010).

What is claimed is:

1. A method of treating obesity in a subject in need thereof, the method comprising administering to the subject a pharmaceutical composition comprising an effective amount of a large amino acid transporter small subunit1 (LAT1) inhibitor.

2. A method of reducing body weight in a subject in need thereof, the method comprising administering to the subject a pharmaceutical composition comprising an effective amount of a large amino acid transporter small subunit1 (LAT1) inhibitor.

3. A method of inhibiting weight gain in a subject in need thereof, the method comprising administering to the subject a pharmaceutical composition comprising an effective amount of a large amino acid transporter small subunit1 (LAT1) inhibitor.

4. A method of reducing white adipose tissue in a subject in need thereof, the method comprising administering to the subject a pharmaceutical composition comprising an effective amount of a large amino acid transporter small subunit1 (LAT1) inhibitor.

5. A method of preventing an increase in white adipose tissue in a subject in need thereof, the method comprising administering to the subject a pharmaceutical composition comprising an effective amount of a large amino acid transporter small subunit1 (LAT1) inhibitor.

6. A method of slowing or preventing the development of diet-associated metabolic syndrome in a subject in need thereof, the method comprising administering to the subject a pharmaceutical composition comprising an effective amount of a large amino acid transporter small subunit1 (LAT1) inhibitor. 7 The method of any one of claim 1, 3, or 5, wherein the obesity, weight gain, or increase in white adipose tissue is diet-associated.

8. The method of claim 7, wherein the obesity, weight gain, or increase in white adipose tissue is associated with a high-fat diet.

9. The method of claim 4 or 5, wherein the reduction in white adipose tissue or the prevention in the increase in white adipose tissue is in epididymal white adipose tissue, inguinal white adipose tissue, or a combination thereof.

10. The method of claim 6, wherein the slowing or prevention in the development of metabolic syndrome comprises a slowing or prevention in the development of metabolic disorders associated with metabolic syndrome.

11. The method of claim 10, wherein the metabolic disorder is dyslipidemia, hypertension, impaired glucose tolerance, compensatory hyperinsulinemia, the tendency to develop fat around the abdomen, or a combination thereof.

12. The method of any of claims 1-11, wherein the pharmaceutical composition is administered systemically.

13. The method of claim 12, wherein the route of systemic administration is selected from intravenously, orally, intraperitoneally, intramuscularly, intradermally, intrathecally, subcutaneously and nasally.

14. The method of any of claims 1-11, wherein the pharmaceutical composition is administered locally to adipose tissue.

15. The method of any of claims 1-14, wherein the subject is a mammal.

16. The method of claim 15, wherein the mammal is human.

17. A method of reducing expression of leptin in an adipocyte, the method comprising contacting the adipocyte with a large amino acid transporter small subunit1 (LAT1) inhibitor.

18. A method of reducing inflammation in adipose tissue, the method comprising contacting the adipose tissue with a large amino acid transporter small subunit1 (LAT1) inhibitor.

19. A method of inducing or increasing expression of one or more brown adipose tissue markers in white adipose tissue, the method comprising contacting the white adipose tissue with a large amino acid transporter small subunit1 (LAT1) inhibitor.

20. The method of claim 19, wherein the one or more brown adipose tissue markers is selected from the group consisting of uncoupling protein 1, transmembrane protein 26, fibroblast growth factor 21, peroxisome proliferator-activated receptor gamma co-activator-1 alpha, and any combination thereof.

21. A methods of inducing conversion of white adipose tissue to brown adipose tissue, the method comprising contacting the white adipose tissue with a large amino acid transporter small subunit1 (LAT1) inhibitor. 22 The method of any one of claims 1-21, wherein the LAT1 inhibitor is JPH203 or OKY-034.

23. The method of any one of claims 1-22, wherein the LAT1 inhibitor is modified to promote the LAT1 inhibitor to target or enter adipose tissue, white adipose tissue, or an adipocyte.

24. A large amino acid transporter small subunit1 inhibitor or pharmaceutical composition thereof, for use in any of the methods of claims 1-23.
